(19) United States
(12) Patent Application Publication
(10) Pub. No.: US 2023/0399380 A1
(43) Pub. Date: Dec. 14, 2023

(54) Methods and Compositions for Treating Aging-Associated Impairments with TIMP-2 Recombinant Proteins

(71) Applicants: Alkahest, Inc., San Carlos, CA (US); Grifols Diagnostic Solutions Inc., Emeryville, CA (US)

(72) Inventors: Jody Berry, Bath, PA (US); Elizabeth Booth, San Leandro, CA (US); Rachel Britton, San Carlos, CA (US); Meghan Kerrisk Campbell, San Francisco, CA (US); Eva Czirr, Foster City, CA (US); Charles Holz, Martinez, CA (US); Tristan Wasley, Alameda, CA (US)

(21) Appl. No.: 18/144,930

(22) Filed: May 9, 2023

Related U.S. Application Data
(60) Provisional application No. 63/343,040, filed on May 17, 2022.

(51) Int. Cl. C07K 14/81; C07K 14/55; C12N 15/63; A61P 25/28

(52) U.S. Cl. CPC C07K 14/8146; C07K 14/55; C12N 15/63; A61P 25/28; C07K 2319/30

(57) ABSTRACT

Methods and compositions for treating and/or preventing aging-related conditions are described. The compositions used in the methods include recombinant protein constructs employing human TIMP-2 protein for use in treating indications related to aging-related cognitive impairment.

### CROSS-REFERENCE TO RELATED APPLICATIONS

Pursuant to 35 U.S.C. § 119(e), this application claims priority to the filing date of: U.S. Provisional Patent Application No. 63/343,040 filed May 17, 2022; the disclosure of which application is herein incorporated by reference.

### 1. INTRODUCTION

a. Aging

The aging nervous system undergoes structural and neurophysiological changes over time such as synapse loss and loss of neuronal function. These changes have also been shown to be associated with cognitive decline. (Hedden T, et al., Nat. Rev. Neurosci., 5: 87-96 (2004)). Aging is also a primary risk factor for dementia-related neurodegenerative disease and neuroinflammation (e.g., Alzheimer's disease (AD), Parkinson's disease (PD), amyotrophic lateral sclerosis (ALS)) for which the prevalence is continuously increasing (Wyss-Coray A, et al., Nature 539:180-86 (2016)). Additionally, neurodegeneration accompanies either primarily or secondarily other neurological diseases such as diseases of demyelination, ischemia, multiple sclerosis (MS), stroke, and traumatic brain injury (TBI). Mayne K, et al., Frontier Aging Neurosci. 12(572090) (2020).

As human lifespan increases, a greater fraction of the population suffers from aging associated cognitive impairments, making it crucial to elucidate means by which to maintain cognitive integrity by protecting against, or even counteracting, the effects of aging (Hebert et al., Arch. Neurol. 60:1119-22 (2003); and Bishop et al., Nature 464:529-35 (2010)). One route that can lead to understanding the changes that occur in aged individuals and thus may cause or correlate with cognitive decline, dementia-related disease, neurodegeneration, or neuroinflammation is to investigate the changes in the plasma proteome between relatively young and old populations of individuals. For example, it is known that plasma from young mice can reverse age-related impairments in synaptic plasticity and cognitive function in aged mice (Villeda S A, et al., Nat. Med. 20(6):659-63 (2014)).

b. TIMP-2

Investigations have been performed to identify factors in plasma that are changed in abundance in a population over the lifespan (Lehallier B, et al., Nat. Med. 25(12):1843-50 (2019)). Proteins comprising middle and old age waves of variation overlap with proteins that have been associated with cognitive impairments and are amplified in diseases of cognitive decline such as Alzheimer's disease (Id.) One such protein that has been identified is tissue inhibitor of metalloprotease 2 (TIMP-2) (Castellano J M, et al., Nature 544(7651):488-92 (2017)). TIMP-2 is a low molecular weight protease inhibitor capable of inhibiting matrix metalloproteases (MMPs), preferentially complexing with the pro-enzyme form of MMP-2 (Emmert-Buck M R, et al., FEBS Letters 364:28-31 (1995)).

MMPs are a family of calcium or zinc-dependent endopeptidases involved in degradation of the extracellular matrix and tissue remodeling. They are also implicated in many disease states including arthritis, tumor invasion, and involvement in inflammatory processes. TIMP proteins form complexes with MMPs, blocking the enzymatic binding pocket. Inhibition is regulated by the TIMP2 N-terminus while binding is primarily regulated by the TIMP2 C-terminus. TIMP proteins' N-terminal domains are highly conserved while the C-terminal domains differ.

TIMP-2 has been shown to provide cognitive learning and memory benefits in aged mice (International Patent Application No. PCT/US2016/036032 which is incorporated by reference herein in its entirety). It has also been shown to be necessary for spatial memory in mice. (Id.) Thus, TIMP-2 is an intriguing therapeutic candidate for the treatment of cognitive decline as it has effects on synaptic plasticity, learning, memory, and other cognitive abilities associated with normal brain aging, dementia, cognitive disease or neurodegenerative disease.

### 2. SUMMARY

Methods and compositions of treating an adult mammal for an aging-associated condition are provided. Aspects of the compositions include recombinant proteins constructed using and modifying the human TIMP2 protein as disclosed in SEQ ID NO:1 through SEQ ID NO: 38 inclusive (detailed below). Aspects of the methods include administering TIMP-2 or TIMP-2 recombinant protein constructs to the mammal in a manner sufficient to treat the mammal for the aging-associated impairment. A variety of aging-associated impairments may be treated by practice of the methods. Such impairments include, for example, cognitive impairments, neurodegeneration, or neuroinflammation.

Although TIMP2 has been previously reported to improve cognitive performance in vivo, the subject compositions provide recombinant TIMP2 proteins with substantially improved features for use in alleviating aging-related cognitive disease and associated impairments. Some such improved features of the subject compositions are a longer half-life as well as equivalent efficacy to native TIMP2 with potential fewer side effects observed with matrix metalloprotease inhibitors.

### 3. BRIEF DESCRIPTION OF THE FIGURES

FIG. 1 is a sodium dodecyl sulfate polyacrylamide gel electrophoresis (SDS-PAGE) image of a purified TIMP2 recombinant protein construct hIL2-TIMP2(27-220) (SEQ ID NO: 1) which substitutes hIL-2 signal peptide (SEQ ID NO: 23) for the endogenous signal peptide of human TIMP2 (1-26, SEQ ID NO: 24). “+BME” indicates columns/wells in which beta-mercaptoethanol was used while “−BME” indicates columns/wells in which beta-mercaptoethanol was not used.

FIG. 2 is a sodium dodecyl sulfate polyacrylamide gel electrophoresis (SDS-PAGE) image of a purified TIMP2 recombinant protein construct hIL2-Ala-TIMP2(27-220) (SEQ ID NO:3). This construct also substitutes hIL-2 signal peptide (SEQ ID NO: 23) for the endogenous signal peptide of human TIMP2 (1-26, SEQ ID NO: 24). “+BME” indicates columns/wells in which beta-mercaptoethanol was used while “−BME” indicates columns/wells in which beta-mercaptoethanol was not used.

FIG. 3 is a sodium dodecyl sulfate polyacrylamide gel electrophoresis (SDS-PAGE) image of a purified TIMP2 recombinant protein construct hIL2-TIMP2(27-152) (SEQ ID NO: 2) where the C-terminal 68 amino acids of full length TIMP2 have been removed. This construct also substitutes human IL-2 signal peptide (SEQ ID NO: 23) for the endogenous signal peptide of human TIMP2 (1-26, SEQ ID NO: 24). “+BME” indicates columns/wells in which beta-mercaptoethanol was used while “−BME” indicates columns/wells in which beta-mercaptoethanol was not used.

FIG. 4 is a sodium dodecyl sulfate polyacrylamide gel electrophoresis (SDS-PAGE) image of several purified TIMP2 recombinant protein constructs. Like the aforementioned constructs in FIGS. 1-3, these constructs also substitute hIL-2 signal peptide (SEQ ID NO: 23) for the endogenous signal peptide of human TIMP2 (1-26, SEQ ID NO: 24). The TIMP2 recombinant protein constructs include: hIL2-8×His-TIMP2(27-220) (SEQ ID NO: 5, Column/well 2); hIL2-TIMP2(27-220)-8×His (SEQ ID NO:6, Column/well 3); hIL2-IgG4(CH2-CH3)-TIMP2(27-220) (SEQ ID NO: 7, Column/well 4); and hIL2-TIMP2(27-220)-IgG4(CH2-CH3) (SEQ ID NO: 8, Column/well 5). Left-handed columns/wells 1-5 contained beta-mercaptoethanol (BME+) and right-handed columns/wells 1-5 were absent beta-mercaptoethanol (BME−) in the sample buffer.

FIG. 5 is a sodium dodecyl sulfate polyacrylamide gel electrophoresis (SDS-PAGE) image of several purified TIMP2 recombinant protein constructs. These constructs also substitute hIL-2 signal peptide (SEQ ID NO: 23) for the endogenous signal peptide of human TIMP2 (1-26, SEQ ID NO: 24). The TIMP2 protein constructs include: hIL2-TIMP2(27-220)-IgG4(hinge-CH2-CH3) (SEQ ID NO: 9, Column/well 2); hIL2-TIMP2(27-152)-8×His (SEQ ID NO: 10, Column/well 3); and hIL2-TIMP2(27-155)-IgG4(CH2-CH3) (SEQ ID NO: 12, Column/well 4). Left-handed columns/wells 1-4 contained beta-mercaptoethanol (BME+) and right-handed columns/wells 1-4 were absent beta-mercaptoethanol (BME−) in the sample buffer.

FIG. 6 is a sodium dodecyl sulfate polyacrylamide gel electrophoresis (SDS-PAGE) image of several purified TIMP2 recombinant protein constructs. These constructs also substitute hIL-2 signal peptide (SEQ ID NO: 23) for the endogenous signal peptide of human TIMP2 (1-26, SEQ ID NO: 24). The TIMP2 protein constructs include: hIL2-TIMP2(153-220)-IgG4(hinge-CH2-CH3) (SEQ ID NO: 13, Column/well 2); hIL2-IgG4 (SEQ ID NO: 14, Column/well 3); and hIL2-IgG4(hinge-CH2-CH3) (SEQ ID NO: 15, Column/well 4). Left-handed columns/wells 1-4 contained beta-mercaptoethanol (BME+) and right-handed columns/wells 1-4 were absent beta-mercaptoethanol (BME−) in the sample buffer.

FIG. 7 is a sodium dodecyl sulfate polyacrylamide gel electrophoresis (SDS-PAGE) image of several purified TIMP2 recombinant protein constructs using the endogenous human TIMP2 signal peptide (SEQ ID NO: 24). These protein constructs include: TIMP2(1-220) (SEQ ID NO: 17, Columns/wells 1 and 7); TIMP2(C27A) (SEQ ID NO: 18, Columns 2 and 8); TIMP2(1-220)-8×His (SEQ ID NO: 19, Columns/wells 3 and 9); and TIMP2(1-220)-IgG4 (SEQ ID NO: 20, Columns/wells 5 and 10). Columns 1-5 contained beta-mercaptoethanol (BME+) and right-handed columns 1-5 were absent beta-mercaptoethanol (BME−).

FIG. 8 displays an SDS-PAGE image of purified and reduced Ala-TIMP2(27-220)-hIgG4(CH2-CH3) (SEQ ID NO: 34).

FIG. 9 displays an SDS-PAGE image of purified and reduced TIMP2(1-220)-mIgG1(CH2-CH3) (SEQ ID NO: 22).

FIG. 10 is a table characterizing data for different lots of TIMP2 proteins with endogenous signal peptide (SEQ ID NO: 24). Row 1 shows characterization of SEQ ID NO: 17. Row 2 shows characterization of SEQ ID NO: 35. Row 3 shows characterization of SEQ ID NO: 14. Row 4 shows characterization of SEQ ID NO: 36. Row 5 shows characterization of SEQ ID NO: 37. Row 6 shows characterization of SEQ ID NO: 22. Row 7 shows characterization of SEQ ID NO: 38. Row 8 shows characterization of SEQ ID NO: 19. The table summarizes from LC/MS intact mass characterization and SEC-MALS to evaluate aggregation state and oligomerization state.

FIGS. 11A to 11D show BLI studies performed on the Octet Red96e in order to characterize binding interactions with matrix metalloproteases (MMPs). TIMP2 and Ala-TIMPI2-hIgG4 were captured using ProA Octet tips or streptavidin Octet tips depending on biotinylation state. MMPs (22.5 nM) were associated at captured biotinylated proteins. Recombinant MMPs were sourced mouse and human. FIG. 11A shows that biotinylated TIMP2 (45 nM) was captured by streptavidin Octet tips. FIG. 11B is a zoomed-in view of the chromatogram in FIG. 11A depicting observed binding curves of MMP3 and MMP9. FIG. 11C shows that biotinylated Ala-TIMP2 (45 nM) was captured by streptavidin Octet tips.

FIGS. 12A to 12C also show BLI studies performed on the Octet Red96e in order to characterize binding interactions with matrix metalloproteases (MMPs). TIMP2 and Ala-TIMPI2-hIgG4 were captured using ProA Octet tips or streptavidin Octet tips depending on biotinylation state. MMPs (22.5 nM) were associated at captured biotinylated proteins. Recombinant MMPs were sourced mouse and human. FIG. 12A shows that biotinylated TIMP2 (45 nM) was captured by streptavidin Octet tips showing association with MMP2. FIG. 12B shows that biotinylated TIMP2 (45 nM) was captured by streptavidin Octet tips showing association with MMP3. FIG. 12C shows that biotinylated TIMP2 (45 nM) was captured by streptavidin Octet tips showing association with MMP9. Ala-TIMP2 (45 nM) was captured by streptavidin Octet tips.

FIG. 13 shows binding interactions of TIMP2 recombinant protein constructs with α3β1 integrin. TIMP2 and Ala-TIMP2 were biotinylated with a 1:1 molar ratio of biotin to protein. TIMP2-hIgG4 and Ala-TIMP2-hIgG4 were biotinylated with a 1:1 molar ratio of biotin to dimerized protein. α3β1 integrin was associated at 22.5 nM to captured biotinylated protein. Binding observed for α3β1 integrin. BLI studies were performed on the Octet Red96e. Biotinylated TIMP2, Ala-TIMP2, TIMP2-hIgG4, and Ala-TIMP2-hIgG4 dimer were captured by Streptavidin Octet Tips at 45 nM. All curves are reference-subtracted.

FIG. 14 is a summary table of association studies from BLI. “−” indicates no observable association; “+” indicates association corresponding to a nanometer shift between 0 and 0.05; “++” indicates an association corresponding to a nanometer shift between 0.05 and 0.2; “+++” indicates an association corresponding to a nanometer shift greater than 0.2. Association ranges were determined by visual inspection of naturally occurring groups. (Abbreviations: recombinant mouse Matrix Metalloproteinase (rmMMP); recombinant human Matrix Metalloproteinase (rhMMP); human Matrix Metalloproteinase (Alk-MMP)).

FIG. 15A reports the results of a determination of the half-lives of TIMP2, Ala-TIMP2, and Ala-TIMP2-hIgG4 recombinant proteins in aged C57BL/6 mice. FIG. 15B reports the results of a determination of the half-lives of TIMP2 and TIMP2-hIgG4. The hIgG4Fc fusion to the TIMP2 protein extended the half-life. Based on this data, it was determined that TIMP2 requires daily dosing while hIgG4 fusion proteins can be dosed once every 3 days.

FIG. 16 shows a table reporting the MMP2, MMP3, and MMP9 inhibitory activity reported as IC₅₀ for certain recombinant TIMP2 constructs as determined by a colorimetric enzymatic activity assay for each MMP performed with recombinant proteins. TIMP2 and TIMP2-IgG4 inhibition was confirmed for MMP2, MMP3, and MMP9. However, the inhibitory function of Ala-TIMP2 fusions was significantly reduced by approximately 1000-fold.

FIG. 17 depicts a plot of % MMP2 activity versus log concentration of TIMP2, Ala-TIMP2, and TIMP2-hIgG4. IC₅₀ values were calculated, confirming TIMP2 and TIMP2-IgG4 inhibition of MMP2 while Ala-TIMP2 displayed over 1000-fold less efficacy at inhibiting MMP2.

FIG. 18 shows that aged C57BL/6 mice treated with TIMP2, Ala-TIMP2, and TIMP2-hIgG4 recombinant proteins were able to remember the novel arm of a Y-maze as measured by percent entries into the novel arm compared to 50% chance. However, Vehicle, hIgG4 control, and Ala-TIMP2-hIgG4 treated mice could not distinguish between the arms. This data was surprising since it is understood that TIMP2's role inhibiting MMPs is expected to play an important part in TIMP2's ability to improve cognitive performance.

FIG. 19 shows the results of a nesting score study performed on aged C57BL/6 mice treated with recombinant TIMP2 and TIMP2-hIgG4. Administration of both constructs resulted in significant improvement in nesting compared to vehicle control.

FIG. 20 shows the results of a nesting score study performed on aged C57BL/6 mice treated with recombinant TIMP2 and Ala-TIMP2. Administration of Ala-TIMP2 did not result in improved nesting scores.

FIG. 21 shows the effects of TIMP2 and TIMP2-hIgG4 recombinant protein administration on the number of excitatory synapses in the CA1 hippocampal region of aged C57BL/6 mice. Excitatory synapses were significantly increased in the CA1 with TIMP2-hIgG4 treatment.

FIG. 22 shows the effects of TIMP2 and TIMP2-hIgG4 recombinant protein administration on the number of excitatory synapses in the dentate gyrus (DG) of aged C57BL/6 mice. Excitatory synapses in the DG were significantly increased with either TIMP2 or TIMP2-hIgG4 treatment.

FIG. 23 shows the effects of TIMP2 and Ala-TIMP2 recombinant protein administration on the number of excitatory synapses in the CA1 hippocampal region of aged C57BL/6 mice. Excitatory synapses in the CA1 were significantly increased with TIMP2 but not Ala-TIMP2 treatment.

FIG. 24 shows the effects of TIMP2 and Ala-TIMP2 recombinant protein administration on the number of excitatory synapses in the dentate gyrus (DG) of aged C57BL/6 mice. Excitatory synapses were increased in the DG with both TIMP2 and Ala-TIMP2 treatment. This is unexpected since it is understood that the effect of TIMP2 on improving cognitive function through increasing excitatory synapse number is dependent on TIMP2's function as an MMP inhibitor, and Ala-TIMP2 is over 1000-fold less effective at inhibiting MMPs than TIMP2.

FIG. 25 shows the results of a study on TIMP2 and TIMP2-hIgG4 brain penetrance in aged C57BL/6 mice. Although brain penetrance of TIMP2 had been previously reported, TIMP2-hIgG4 also penetrated the brain. This is unexpected as there are known, active mechanisms that prevent IgG and IgG fusion proteins from penetrating the brain in appreciable amounts.

### 4. INCORPORATION BY REFERENCE

All publications and patent applications mentioned in this specification are herein incorporated by reference to the same extent as if each individual publication or patent application was specifically and individually indicated to be incorporated by reference

### 5. DETAILED DESCRIPTION

Methods of treating an adult mammal for an aging-associated impairment are provided. Aspects of the methods include administering the TIMP2 recombinant proteins to the mammal in a manner sufficient to treat the mammal for the aging-associated impairment. A variety of aging-associated impairments may be treated by practice of the methods, which impairments include cognitive impairments, neurodegeneration, and neuroinflammation.

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

a. Compositions

In some embodiments compositions comprised of fusion proteins related to human TIMP2 protein are provided. In some embodiments the fusion protein comprises a recombinant TIMP2 protein fused to additional polypeptide sequences. In some embodiments the additional polypeptide sequences are comprised of a signal peptide or leader sequence. Said signal peptide or leader sequence in some embodiments is comprised of the endogenous leader sequence of human interleukin-2 (IL-2) (SEQ ID NO: 23). However, said signal peptide or leader sequence in other embodiments may be comprised of signal peptides or leader sequences from a variety of known human or mammalian proteins known to those having ordinary skill in the art, including the endogenous signal peptide/leader sequence of human TIMP2.

In additional embodiments the other polypeptide sequences can be comprised of the sequences listed below as SEQ ID NOs:1 through 38 inclusive. Further embodiments of the additional polypeptide sequences that can be fused to human TIMP2 include polypeptides with 90%, 91%, 92%, 93%, 94%, 95%, 96%, 97%, 98%, 99%, or 100% sequence identity to SEQ ID NOs: 1 through 38 inclusive.

In further embodiments the additional polypeptide sequences fused to a recombinant TIMP2 polypeptide sequence can comprise the sequence of an immunoglobulin Fc region that has sequence identity 90% identity to the hIgG4 sequence listed below. In another embodiment the immunoglobulin Fc region sequences can have sequence identity of 91%, 92%, 93%, 94%, 95%, 96%, 97%, 98%, 99%, or 100% sequence identity to the immunoglobulin Fc region sequences listed below (e.g., SEQ ID NOs: 28 and 29). Said immunoglobulin Fc regions may be used to increase the half-life of the fused TIMP2 construct in vivo.

In other embodiments the additional polypeptide sequences fused to a recombinant TIMP2 polypeptide may be placed N-terminally or C-terminally to the TIMP2 polypeptide. SEQ ID NOs: 1 through 38 may be fused to the recombinant TIMP2 polypeptide in any combination or number.

In further embodiments the TIMP2 polypeptide may not be full-length (amino acids 1-200 SEQ ID NO: 17) or may be a splice variant known to those have ordinary skill in the art. The TIMP2 polypeptide may be a truncated version of SEQ ID NO: 17. For example, it may be lacking its native signal peptide or other known functional regions such as polypeptide regions known for binding and/or inhibiting the function of matrix metalloproteases (MMPs). Another embodiment of the fused TIMP2 polypeptide comprises a TIMP2 polypeptide with an alanine inserted before amino acid number 27 of the native TIMP2 protein (SEQ ID NO: 17), such as SEQ ID NOs: 3, 34, 36, and 37 by way of example and not limitation.

hIL2-TIMP2(27-220)
SEQ ID NO: 1
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGcECKITRCPMIPCYISSPDECLWMDWVTEKNING
HQAKFFACIKRSDGSCAWYRGAAPPKQEFLDIEDP
hIL2-TIMP2(27-152)
SEQ ID NO: 2
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGc
hIL2-Ala-TIMP2 (27-220)
SEQ ID NO: 3
MYRMQLLSCIALSLALVTNSACSCSPVHPQQAFcNADVVIRAKAVSEKEVDSGNDIYGN
PIKRIQYEIKQIKMFKGPEKDIEFIYTAPSSAVCGVSLDVGGKKEYLIAGKAEGDGKMHIT
LCDFIVPWDTLSTTQKKSLNHRYQMGcECKITRCPMIPCYISSPDECLWMDWVTEKNIN
GHQAKFFACIKRSDGSCAWYRGAAPPKQEFLDIEDP
hIL2-TIMP2(153-220)
SEQ ID NO: 4
MYRMQLLSCIALSLALVTNSECKITRCPMIPCYISSPDECLWMDWVTEKNINGHQAKFF
ACIKRSDGSCAWYRGAAPPKQEFLDIEDP
hIL2-8xHis-TIMP2 (27-220)
SEQ ID NO: 5
MYRMQLLSCIALSLALVTNShhhhhhhhCSCSPVHPQQAFcNADVVIRAKAVSEKEVDSGN
DIYGNPIKRIQYEIKQIKMFKGPEKDIEFIYTAPSSAVCGVSLDVGGKKEYLIAGKAEGDG
KMHITLCDFIVPWDTLSTTQKKSLNHRYQMGcECKITRCPMIPCYISSPDECLWMDWVT
EKNINGHQAKFFACIKRSDGSCAWYRGAAPPKQEFLDIEDP
hIL2-TIMP2 (27-220)-8xHis
SEQ ID NO: 6
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGcECKITRCPMIPCYISSPDECLWMDWVTEKNING
HQAKFFACIKRSDGSCAWYRGAAPPKQEFLDIEDPhhhhhhhh
hI12-IgG4 (CH2-CH3)-TIMP2 (27-220)
SEQ ID NO: 7
MYRMQLLSCIALSLALVTNSSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTT
PPVLDSDGSFFLYSRLTVDKSRWQQGNVFSCSVMHEALHNHYTQKSLSLSCSCSPVHPQ
QAFcNADVVIRAKAVSEKEVDSGNDIYGNPIKRIQYEIKQIKMFKGPEKDIEFIYTAPSSA
VCGVSLDVGGKKEYLIAGKAEGDGKMHITLCDFIVPWDTLSTTQKKSLNHRYQMGcEC
KITRCPMIPCYISSPDECLWMDWVTEKNINGHQAKFFACIKRSDGSCAWYRGAAPPKQE
FLDIEDP
hIL2-TIMP2 (27-220)-IgG4 (CH2-CH3)
SEQ ID NO: 8
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGcECKITRCPMIPCYISSPDECLWMDWVTEKNING
HQAKFFACIKRSDGSCAWYRGAAPPKQEFLDIEDPSVFLFPPKPKDTLMISRTPEVTCVV
VDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQQGNVFSCSVMHEALHNHYTQ
KSLSLS
hIL2-TIMP2 (27-220)-IgG4 (hinge-CH2-CH3)
SEQ ID NO: 9
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGcECKITRCPMIPCYISSPDECLWMDWVTEKNING
HQAKFFACIKRSDGSCAWYRGAAPPKQEFLDIEDPESKYGPPCPPCPAPEFLGGPSVFLFP
PKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQQG
NVFSCSVMHEALHNHYTQKSLSL
hIL2-TIMP2 (27-152)-8xHis
SEQ ID NO: 10
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGchhhhhhhh
hIL2-TIMP2 (153-220)-8xHis
SEQ ID NO: 11
MYRMQLLSCIALSLALVTNSECKITRCPMIPCYISSPDECLWMDWVTEKNINGHQAKFF
ACIKRSDGSCAWYRGAAPPKQEFLDIEDPhhhhhhhh
hIL2-TIMP2 (27-155)-IgG4 (CH2-CH3)
SEQ ID NO: 12
MYRMQLLSCIALSLALVTNSCSCSPVHPQQAFcNADVVIRAKAVSEKEVDSGNDIYGNPI
KRIQYEIKQIKMFKGPEKDIEFIYTAPSSAVCGVSLDVGGKKEYLIAGKAEGDGKMHITL
CDFIVPWDTLSTTQKKSLNHRYQMGcGVSSVFLFPPKPKDTLMISRTPEVTCVVVDVSQE
DPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNG
QPENNYKTTPPVLDSDGSFFLYSRLTVDKSRWQQGNVFSCSVMHEALHNHYTQKSLSL
S
hIL2-TIMP2 (153-220)-IgG4 (CH2-CH3)
SEQ ID NO: 13
MYRMQLLSCIALSLALVTNSECKITRCPMIPCYISSPDECLWMDWVTEKNINGHQAKFF
ACIKRSDGSCAWYRGAAPPKQEFLDIEDPSVFLFPPKPKDTLMISRTPEVTCVVVDVSQE
DPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNG
QPENNYKTTPPVLDSDGSFFLYSRLTVDKSRWQQGNVFSCSVMHEALHNHYTQKSLSL
S
hIL2-IgG4 (CH2-CH3)
SEQ ID NO: 14
MYRMQLLSCIALSLALVTNSSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTT
PPVLDSDGSFFLYSRLTVDKSRWQQGNVFSCSVMHEALHNHYTQKSLSLS
hIL2-IgG4 (hinge-CH3-CH4)
SEQ ID NO: 15
MYRMQLLSCIALSLALVTNSESKYGPPCPPCPAPEFLGGPSVFLFPPKPKDTLMISRTPEV
TCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQQGNVFSCSVMHEALHN
HYTQKSLSL
hIL2-IgG4 (hinge-CH3-CH4) N-terminal serine
SEQ ID NO: 16
MYRMQLLSCIALSLALVTNSESKYGPPCPPCPAPEFLGGPSVFLFPPKPKDTLMISRTPEV
TCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQQGNVFSCSVMHEALHN
HYTQKSLSLS
TIMP2 (1-220)
SEQ ID NO: 17
MGAAARTLRLALGLLLLATLLRPADACSCSPVHPQQAFcNADVVIRAKAVSEKEVDSG
NDIYGNPIKRIQYEIKQIKMFKGPEKDIEFIYTAPSSAVCGVSLDVGGKKEYLIAGKAEGD
GKMHITLCDFIVPWDTLSTTQKKSLNHRYQMGcECKITRCPMIPCYISSPDECLWMDWV
TEKNINGHQAKFFACIKRSDGSCAWYRGAAPPKQEFLDIEDP
TIMP2 (C27A)
SEQ ID NO: 18
MGAAARTLRLALGLLLLATLLRPADAaSCSPVHPQQAFcNADVVIRAKAVSEKEVDSGN
DIYGNPIKRIQYEIKQIKMFKGPEKDIEFIYTAPSSAVCGVSLDVGGKKEYLIAGKAEGDG
KMHITLCDFIVPWDTLSTTQKKSLNHRYQMGcECKITRCPMIPCYISSPDECLWMDWVT
EKNINGHQAKFFACIKRSDGSCAWYRGAAPPKQEFLDIEDP
TIMP2 (1-220)-8xHis
SEQ ID NO: 19
MGAAARTLRLALGLLLLATLLRPADACSCSPVHPQQAFcNADVVIRAKAVSEKEVDSG
NDIYGNPIKRIQYEIKQIKMFKGPEKDIEFIYTAPSSAVCGVSLDVGGKKEYLIAGKAEGD
GKMHITLCDFIVPWDTLSTTQKKSLNHRYQMGcECKITRCPMIPCYISSPDECLWMDWV
TEKNINGHQAKFFACIKRSDGSCAWYRGAAPPKQEFLDIEDPhhhhhhhh
TIMP2 (1-220)-IgG4
SEQ ID NO: 20
MGAAARTLRLALGLLLLATLLRPADACSCSPVHPQQAFcNADVVIRAKAVSEKEVDSG
NDIYGNPIKRIQYEIKQIKMFKGPEKDIEFIYTAPSSAVCGVSLDVGGKKEYLIAGKAEGD
GKMHITLCDFIVPWDTLSTTQKKSLNHRYQMGcECKITRCPMIPCYISSPDECLWMDWV
TEKNINGHQAKFFACIKRSDGSCAWYRGAAPPKQEFLDIEDPSVFLFPPKPKDTLMISRTP
EVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQQGNVFSCSVMHEAL
HNHYTQKSLSLS
TIMP2 (1-220)-mIgG2a
SEQ ID NO: 21
MGAAARTLRLALGLLLLATLLRPADACSCSPVHPQQAFCNADVVIRAKAVSEKEVDSG
NDIYGNPIKRIQYEIKQIKMFKGPEKDIEFIYTAPSSAVCGVSLDVGGKKEYLIAGKAEGD
GKMHITLCDFIVPWDTLSTTQKKSLNHRYQMGCECKITRCPMIPCYISSPDECLWMDWV
TEKNINGHQAKFFACIKRSDGSCAWYRGAAPPKQEFLDIEDPPSVFIFPPKIKDVLMISLS
PIVTCVVVDVSEDDPDVQISWFVNNVEVHTAQTQTHREDYNSTLRVVSALPIQHQDWM
SGKEFKCKVNNKDLPAPIERTISKPKGSVRAPQVYVLPPPEEEMTKKQVTLTCMVTDFM
PEDIYVEWTNNGKTELNYKNTEPVLDSDGSYFMYSKLRVEKKNWVERNSYSCSVVHEG
LHNHHTTKSFSRTPGK
TIMP2(1-220)-mIgG1(CH2-CH3)
SEQ ID NO: 22
MGAAARTLRLALGLLLLATLLRPADACSCSPVHPQQAFCNADVVIRAKAVSEKEVDSG
NDIYGNPIKRIQYEIKQIKMFKGPEKDIEFIYTAPSSAVCGVSLDVGGKKEYLIAGKAEGD
GKMHITLCDFIVPWDTLSTTQKKSLNHRYQMGCECKITRCPMIPCYISSPDECLWMDWV
TEKNINGHQAKFFACIKRSDGSCAWYRGAAPPKQEFLDIEDPVPEVSSVFIFPPKPKDVLT
ITLTPKVTCVVVDISKDDPEVQFSWFVDDVEVHTAQTQPREEQFNSTFRSVSELPIMHQD
WLNGKEFKCRVNSAAFPAPIEKTISKTKGRPKAPQVYTIPPPKEQMAKDKVSLTCMITDF
FPEDITVEWQWNGQPAENYKNTQPIMNTNGSYFVYSKLNVQKSNWEAGNTFTCSVLHE
GLHNHHTEKSLSHSPGK
hIL2 Signal Peptide
SEQ ID NO: 23
MYRMQLLSCIALSLALVINS
TIMP2 Signal Peptide (1-26)
SEQ ID NO: 24
MGAAARTLRLALGLLLLATLLRPADA
TIMP2 (27-220)
SEQ ID NO: 25
CSCSPVHPQQAFcNADVVIRAKAVSEKEVDSGNDIYGNPIKRIQYEIKQIKMFKGPEKDIE
FIYTAPSSAVCGVSLDVGGKKEYLIAGKAEGDGKMHITLCDFIVPWDTLSTTQKKSLNH
RYQMGcECKITRCPMIPCYISSPDECLWMDWVTEKNINGHQAKFFACIKRSDGSCAWYR
GAAPPKQEFLDIEDP
TIMP2 N-terminal domain (27-152)
SEQ ID NO: 26
CSCSPVHPQQAFcNADVVIRAKAVSEKEVDSGNDIYGNPIKRIQYEIKQIKMFKGPEKDIE
FIYTAPSSAVCGVSLDVGGKKEYLIAGKAEGDGKMHITLCDFIVPWDTLSTTQKKSLNH
RYQMGc
TIMP2 C-terminal domain (153-220)
SEQ ID NO: 27
ECKITRCPMIPCYISSPDECLWMDWVTEKNINGHQAKFFACIKRSDGSCAWYRGAAPPK
QEFLDIEDP
hIgG4 CH2-CH3
SEQ ID NO: 28
SVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKS
RWQQGNVFSCSVMHEALHNHYTQKSLSL
hIgG4 CH2-CH3 N-terminal serine
SEQ ID NO: 29
SVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKS
RWQQGNVFSCSVMHEALHNHYTQKSLSLS
hIgG4 hinge
SEQ ID NO: 30
ESKYGPPCPPCPAPEFLGGP
8xHis
SEQ ID NO: 31
HHHHHHHH
mIgG2a (CH2-CH3)
SEQ ID NO: 32
PSVFIFPPKIKDVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEK
KNWVERNSYSCSVVHEGLHNHHTTKSFSRTPGK
mIgG1 (CH2-CH3)
SEQ ID NO: 33
VPEVSSVFIFPPKPKDVLTITLTPKVTCVVVDISKDDPEVQFSWFVDDVEVHTAQTQPRE
EQFNSTFRSVSELPIMHQDWLNGKEFKCRVNSAAFPAPIEKTISKTKGRPKAPQVYTIPPP
KEQMAKDKVSLTCMITDFFPEDITVEWQWNGQPAENYKNTQPIMNINGSYFVYSKLNV
QKSNWEAGNTFTCSVLHEGLHNHHTEKSLSHSPGK
Ala-TIMP2 (27-220)-hIgG4 (CH2-CH3)
SEQ ID NO: 34
ACSCSPVHPQQAFcNADVVIRAKAVSEKEVDSGNDIYGNPIKRIQYEIKQIKMFKGPEKDI
EFIYTAPSSAVCGVSLDVGGKKEYLIAGKAEGDGKMHITLCDFIVPWDTLSTTQKKSLN
HRYQMGcECKITRCPMIPCYISSPDECLWMDWVTEKNINGHQAKFFACIKRSDGSCAWY
RGAAPPKQEFLDIEDPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLD
SDGSFFLYSRLTVDKSRWQQGNVFSCSVMHEALHNHYTQKSLSL
TIMP2(1-220)-hIgG4Fc (CH2-CH3)
SEQ ID NO: 35
MGAAARTLRLALGLLLLATLLRPADACSCSPVHPQQAFcNADVVIRAKAVSEKEVDSG
NDIYGNPIKRIQYEIKQIKMFKGPEKDIEFIYTAPSSAVCGVSLDVGGKKEYLIAGKAEGD
GKMHITLCDFIVPWDTLSTTQKKSLNHRYQMGcECKITRCPMIPCYISSPDECLWMDWV
TEKNINGHQAKFFACIKRSDGSCAWYRGAAPPKQEFLDIEDPSVFLFPPKPKDTLMISRTP
EVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYP
SDIAVEWESNGQPENNYKTTPPVLDSDGSFFLYSRLTVDKSRWQQGNVFSCSVMHEAL
HNHYTQKSLSL
TIMP2(1-26)-Ala-TIMP2(27-220)
SEQ ID NO: 36
MGAAARTLRLALGLLLLATLLRPADAACSCSPVHPQQAFcNADVVIRAKAVSEKEVDS
GNDIYGNPIKRIQYEIKQIKMFKGPEKDIEFIYTAPSSAVCGVSLDVGGKKEYLIAGKAEG
DGKMHITLCDFIVPWDTLSTTQKKSLNHRYQMGcECKITRCPMIPCYISSPDECLWMDW
VTEKNINGHQAKFFACIKRSDGSCAWYRGAAPPKQEFLDIEDP
TIMP2(1-26)-Ala-TIMP2(27-220)-hIgG4Fc (CH2-CH3)
SEQ ID NO: 37
MGAAARTLRLALGLLLLATLLRPADAACSCSPVHPQQAFcNADVVIRAKAVSEKEVDS
GNDIYGNPIKRIQYEIKQIKMFKGPEKDIEFIYTAPSSAVCGVSLDVGGKKEYLIAGKAEG
DGKMHITLCDFIVPWDTLSTTQKKSLNHRYQMGcECKITRCPMIPCYISSPDECLWMDW
VTEKNINGHQAKFFACIKRSDGSCAWYRGAAPPKQEFLDIEDPSVFLFPPKPKDTLMISR
TPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQQGNVFSCSVMHE
ALHNHYTQKSLSLS
hIL2-mIgG1Fc (CH2-CH3)
SEQ ID NO: 38
MYRMQLLSCIALSLALVTNSVPEVSSVFIFPPKPKDVLTITLTPKVTCVVVDISKDDPEVQ
FSWFVDDVEVHTAQTQPREEQFNSTFRSVSELPIMHQDWLNGKEFKCRVNSAAFPAPIE
KTISKTKGRPKAPQVYTIPPPKEQMAKDKVSLTCMITDFFPEDITVEWQWNGQPAENYK
NTQPIMNTNGSYFVYSKLNVQKSNWEAGNTFTCSVLHEGLHNHHTEKSLSHSPGK

In addition to the specific TIMP proteins described above, homologs or proteins (or fragments thereof) from other species, e.g., other animal species, may also be employed in embodiments of the methods, where such homologs or proteins may be from a variety of different types of species, including animals, such as mammals, e.g., rodents, such as mice, rats; domestic animals, e.g., horse, cow, dog, cat; etc. By homolog is meant a protein having 35% or more, such as 40% and more and including 60% or more amino acid sequence identity to the specific TIMP proteins as identified in SEQ ID NOS: 01 to 04, where sequence identity is determined using BLAST at default settings.

In addition to the TIMP proteins, e.g., as described above, TIMP polypeptides that vary from the TIMP proteins may also be employed in practicing methods of the invention. Different variations may be present, including but not limited to substitution, insertion and/or deletion mutations, as well as other types of non-amino acid sequence variations, e.g., as illustrated below. TIMP polypeptides that may be employed include proteins having an amino acid sequence encoded by an open reading frame (ORF) of a TIMP gene, including the full-length TIMP protein and fragments thereof, such as biologically active fragments and/or fragments corresponding to functional domains; and including fusions of the subject polypeptides to other proteins or parts thereof. Fragments of interest may vary in length, and in some instances are 10 aa or longer, such as 50 aa or longer, and including 100 aa or longer, and in some instances do not exceed 150 aa in length, where a given fragment will have a stretch of amino acids that is substantially the same as or identical to a subsequence found in the sequences provided above; where the subsequence may vary in length and in some instances is 10 aa or longer, such as 15 aa or longer, up to 50 aa or even longer.

In some instances, TIMP polypeptides employed in methods of invention include or more modifications. Modifications that may be present may vary, and include but are not limited to: amide bond substitutions, amino acid substitutions, including of cysteine residues/analogues, cyclization, pegylation, etc. Examples of modifications that may be found in TIMP polypeptides employed in methods of the invention are now reviewed in greater detail.

In some cases, TIMP polypeptides include one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of a TIMP polypeptide can be substituted. In another example, one or more amide linkages (—CO—NH—) in a TIMP polypeptide can be replaced with a linkage which is an isostere of an amide linkage, such as —CH₂NH—, —CH₂S—, —CH₂CH₂—, —CH═CH-(cis and trans), —COCH₂—, —CH(OH)CH₂— or —CH₂SO—. One or more amide linkages in a TIMP polypeptide can also be replaced by, for example, a reduced isostere pseudopeptide bond.

One or more amino acid substitutions can be made in a TIMP polypeptide. The following are non-limiting examples: a) substitution of alkyl-substituted hydrophobic amino acids, including alanine, leucine, isoleucine, valine, norleucine, (S)-2-aminobutyric acid, (S)-cyclohexylalanine or other simple alpha-amino acids substituted by an aliphatic side chain from C₁-C₁₀ carbons including branched, cyclic and straight chain alkyl, alkenyl or alkynyl substitutions; b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from Ci-C4)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine; c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid; d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids; e) substitution of side chain amide residues, including asparagine, glutamine, and alkyl or aromatic substituted derivatives of asparagine or glutamine; and f) substitution of hydroxyl-containing amino acids, including serine, threonine, homoserine, 2,3-diaminopropionic acid, and alkyl or aromatic substituted derivatives of serine or threonine.

In some cases, a TIMP polypeptide includes one or more naturally occurring non-genetically encoded L-amino acids, synthetic L-amino acids, or D-enantiomers of an amino acid. For example, a TIMP polypeptide can include only D-amino acids. For example, a TIMP polypeptide can include one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

A cysteine residue or a cysteine analog can be introduced into a TIMP polypeptide to provide for linkage to another peptide via a disulfide linkage or to provide for cyclization of the TIMP polypeptide. a TIMP polypeptide can be cyclized. One or more cysteines or cysteine analogs can be introduced into a TIMP polypeptide, where the introduced cysteine or cysteine analog can form a disulfide bond with a second introduced cysteine or cysteine analog. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ—CO— or —(CH₂)ₙ—C₆H₄—CO—) with functional groups which allow for the introduction of a bridge. Some examples are disulfides, disulfide mimetics such as the —(CH2)ₙ-carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

Modifications may be present that provide for improvements in one or more physical properties of the TIMP polypeptide. Improvements of physical properties include, for example, modulating immunogenicity; methods of increasing water solubility, bioavailability, serum half-life, and/or therapeutic half-life; and/or modulating biological activity. Examples of such modifications include, but are not limited to: pegylation, glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins. Further details of modifications of interest include those described in U.S. Pat. No. 10,617,744, the disclosure of which is herein incorporated by reference.

In some instances, systemic TIMP polypeptide levels are increased by administering a nucleic acid coding sequence to the subject under conditions sufficient for the coding sequence to be expressed in the subject. Depending on the desired TIMP polypeptide, the nucleic acid coding sequence may vary. Of interest are sequences that encode proteins as described above.

By nucleic acid composition is meant a composition comprising a sequence of DNA having an open reading frame that encodes a TIMP polypeptide of interest, i.e., a TIMP coding sequence, and is capable, under appropriate conditions, of being expressed as a TIMP polypeptide. Also encompassed in this term are nucleic acids that are homologous, substantially similar or identical to the specific nucleic acids described above. In addition to the above described specific nucleic acid compositions, also of interest are homologues of the above sequences. In certain embodiments, sequence similarity between homologues is 20% or higher, such as 25% or higher, and including 30%, 35%, 40%, 50%, 60%, 70% or higher, including 75%, 80%, 85%, 90% and 95% or higher. Sequence similarity is calculated based on a reference sequence, which may be a subset of a larger sequence, such as a conserved motif, coding region, flanking region, etc. A reference sequence may be 18 nt long or longer, such as 30 nt long, and may extend to the complete sequence that is being compared. Algorithms for sequence analysis are known in the art, such as BLAST, described in Altschul et al. (1990), J. Mol. Biol. 215:403-10 (using default settings, i.e., parameters w=4 and T=17). Of particular interest in certain embodiments are nucleic acids of substantially the same length as specific human TIMP1 to TIMP4 nucleic acids mentioned above, where by substantially the same length is meant that any difference in length does not exceed about 20 number %, usually does not exceed about 10 number % and more usually does not exceed about 5 number %; and have sequence identity to any of these sequences of at 90% or greater, such as 95% or greater and including 99% or greater over the entire length of the nucleic acid. In some embodiments, the nucleic acids have a sequence that is substantially similar or identical to the above specific sequences. By substantially similar is meant that sequence identity is 60% or greater, such as 75% or greater and including 80, 85, 90, or even 95% or greater. Nucleic acids of interest also include nucleic acids that encode the proteins encoded by the above described nucleic acids, but differ in sequence from the above described nucleic acids due to the degeneracy of the genetic code.

Nucleic acids as described herein may be present in a vector. Various vectors (e.g., viral vectors, bacterial vectors, or vectors capable of replication in eukaryotic and prokaryotic hosts) can be used in accordance with the present invention. Numerous vectors which can replicate in eukaryotic and prokaryotic hosts are known in the art and are commercially available. In some instances, such vectors used in accordance with the invention are composed of a bacterial origin of replication and a eukaryotic promoter operably linked to a DNA of interest.

Viral vectors used in accordance with the invention may be composed of a viral particle derived from a naturally-occurring virus which has been genetically altered to render the virus replication-defective and to express a recombinant gene of interest in accordance with the invention. Once the virus delivers its genetic material to a cell, it does not generate additional infectious virus but does introduce exogenous recombinant genes into the cell, preferably into the genome of the cell. Numerous viral vectors are well known in the art, including, for example, retrovirus, adenovirus, adeno-associated virus, herpes simplex virus (HSV), cytomegalovirus (CMV), vaccinia and poliovirus vectors.

The DNA of interest may be administered using a non-viral vector, for example, as a DNA- or RNA-liposome complex formulation. Such complexes comprise a mixture of lipids which bind to genetic material (DNA or RNA), providing a hydrophobic coat which allows the genetic material to be delivered into cells. Liposomes which can be used in accordance with the invention include DOPE (dioleyl phosphatidyl ethanol amine), CUDMEDA (N-(5-cholestrum-3-.beta.-ol 3-urethanyl)-N′,N′-dimethylethylene diamine). When the DNA of interest is introduced using a liposome, in some instances one first determines in vitro the optimal values for the DNA: lipid ratios and the absolute concentrations of DNA and lipid as a function of cell death and transformation efficiency for the particular type of cell to be transformed. These values can then be used in or extrapolated for use in in vivo transformation. The in vitro determinations of these values can be readily carried out using techniques which are well known in the art.

Other non-viral vectors may also be used in accordance with the present invention. These include chemical formulations of DNA or RNA coupled to a carrier molecule (e.g., an antibody or a receptor ligand) which facilitates delivery to host cells for the purpose of altering the biological properties of the host cells. By the term “chemical formulations” is meant modifications of nucleic acids to allow coupling of the nucleic acid compounds to a carrier molecule such as a protein or lipid, or derivative thereof. Exemplary protein carrier molecules include antibodies specific to the cells of a targeted secretory gland or receptor ligands, i.e., molecules capable of interacting with receptors associated with a cell of a targeted secretory gland.

DNA constructs may include a promoter to facilitate expression of the DNA of interest within a target cell, such as a strong, eukaryotic promoter. Exemplary eukaryotic promoters include promoters from cytomegalovirus (CMV), mouse mammary tumor virus (MMTV), Rous sarcoma virus (RSV), and adenovirus. More specifically, exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982).

Additional details regarding proteins, nucleic acid coding sequences and vectors for administration of the same are provided in U.S. Pat. No. 10,617,744, the disclosure of which is herein incorporated by reference.

b. Methods

As summarized above, aspects of the invention include methods of treating an aging-associated impairment in an adult mammal. The aging-associated impairment may manifest in a number of different ways, e.g., as aging-associated cognitive impairment and/or physiological impairment, e.g., in the form of damage to central or peripheral organs of the body, such as but not limited to: cell injury, tissue damage, organ dysfunction, aging associated lifespan shortening and carcinogenesis, where specific organs and tissues of interest include, but are not limited to skin, neuron, muscle, pancreas, brain, kidney, lung, stomach, intestine, spleen, heart, adipose tissue, testes, ovary, uterus, liver and bone; in the form of decreased neurogenesis, etc.

In some embodiments, the aging-associated impairment is an aging-associated impairment in cognitive ability in an individual, i.e., an aging-associated cognitive impairment. By cognitive ability, or “cognition,” it is meant the mental processes that include attention and concentration, learning complex tasks and concepts, memory (acquiring, retaining, and retrieving new information in the short and/or long term), information processing (dealing with information gathered by the five senses), visuospatial function (visual perception, depth perception, using mental imagery, copying drawings, constructing objects or shapes), producing and understanding language, verbal fluency (word-finding), solving problems, making decisions, and executive functions (planning and prioritizing). By “cognitive decline”, it is meant a progressive decrease in one or more of these abilities, e.g., a decline in memory, language, thinking, judgment, etc. By “an impairment in cognitive ability” and “cognitive impairment,” it is meant a reduction in cognitive ability relative to a healthy individual, e.g., an age-matched healthy individual, or relative to the ability of the individual at an earlier point in time, e.g., 2 weeks, 1 month, 2 months, 3 months, 6 months, 1 year, 2 years, 5 years, or 10 years or more previously. Aging-associated cognitive impairments include impairments in cognitive ability that are typically associated with aging, including, for example, cognitive impairment associated with the natural aging process, e.g., mild cognitive impairment (M.C.I.); and cognitive impairment associated with an aging associated disorder, that is, a disorder that is seen with increasing frequency with increasing senescence, e.g., a neurodegenerative condition such as Alzheimer's disease, Parkinson's 5 disease, frontotemporal dementia, Huntington's disease, amyotrophic lateral sclerosis, multiple sclerosis, glaucoma, myotonic dystrophy, vascular dementia, and the like. Another embodiment includes an impairment that typically clinically manifests with symptoms or characteristics similar to those observed in aging-associated cognitive impairments yet affect a subject or individual who is younger in age. For example, the age of said subject or individual may be 18 years old or younger, 20 years old or younger, 30 years old or younger, or 39 years old or younger.

By “treatment” it is meant that at least an amelioration of one or more symptoms associated with an aging-associated impairment afflicting the adult mammal is achieved, where amelioration is used in a broad sense to refer to at least a reduction in the magnitude of a parameter, e.g., a symptom associated with the impairment being treated. As such, treatment also includes situations where a pathological condition, or at least symptoms associated therewith, are completely inhibited, e.g., prevented from happening, or stopped, e.g., terminated, such that the adult mammal no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, increased neurogenesis, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

In some instances where the aging-associated impairment is aging-associated cognitive decline, treatment by methods of the present disclosure slows, or reduces, the progression of aging-associated cognitive decline. In other words, cognitive abilities in the individual decline more slowly, if at all, following treatment by the disclosed methods than prior to or in the absence of treatment by the disclosed methods. In some instances, treatment by methods of the present disclosure stabilizes the cognitive abilities of an individual. For example, the progression of cognitive decline in an individual suffering from aging-associated cognitive decline is halted following treatment by the disclosed methods. As another example, cognitive decline in an individual, e.g., an individual 40 years old or older, that is projected to suffer from aging-associated cognitive decline, is prevented following treatment by the disclosed methods. In other words, no (further) cognitive impairment is observed. In some instances, treatment by methods of the present disclosure reduces, or reverses, cognitive impairment, e.g., as observed by improving cognitive abilities in an individual suffering from aging-associated cognitive decline. In other words, the cognitive abilities of the individual suffering from aging-associated cognitive decline following treatment by the disclosed methods are better than they were prior to treatment by the disclosed methods, i.e., they improve upon treatment. In some instances, treatment by methods of the present disclosure abrogates cognitive impairment. In other words, the cognitive abilities of the individual suffering from aging-associated cognitive decline are restored, e.g., to their level when the individual was about 40 years old or less, following treatment by the disclosed methods, e.g., as evidenced by improved cognitive abilities in an individual suffering from aging-associated cognitive decline. In subjects or individuals where cognitive impairment or decline occurs at a younger age (e.g., younger than 40 years old), the cognitive ability is returned to a level where the individual no longer exhibits said cognitive impairment/decline after treatment by the disclosed methods.

In some instances, treatment of an adult mammal in accordance with the methods results in a change in a central organ, e.g., a central nervous system organ, such as the brain, spinal cord, etc., where the change may manifest in a number of different ways, e.g., as described in greater detail below, including but not limited to molecular, structural and/or functional, e.g., in the form of enhanced neurogenesis.

As summarized above, methods described herein are methods of treating an aging associated impairment, e.g., as described above, in an adult mammal. By adult mammal is meant a mammal that has reached maturity, i.e., that is fully developed. As such, adult mammals are not juvenile. Mammalian species that may be treated with the present methods include canines and felines; equines; bovines; ovines; etc., and primates, including humans. The subject methods and compositions, compositions, and reagents may also be applied to animal models, including small mammals, e.g., murine, lagomorpha, etc., for example, in experimental investigations. The discussion below will focus on the application of the subject methods, compositions, reagents, devices and kits to humans, but it will be understood by the ordinarily skilled artisan that such descriptions can be readily modified to other mammals of interest based on the knowledge in the art.

The age of the adult mammal may vary, depending on the type of mammal that is being treated. Where the adult mammal is a human, the age of the human is generally 18 years or older. In some instances, the adult mammal is an individual suffering from or at risk of suffering from an aging-associated impairment, such as an aging-associated cognitive impairment, where the adult mammal may be one that has been determined, e.g., in the form of receiving a diagnosis, to be suffering from or at risk of suffering from an aging associated impairment, such as an aging-associated cognitive impairment. The phrase “an individual suffering from or at risk of suffering from an aging-associated cognitive impairment” refers to an individual that is about 50 years old or older, e.g., 60 years old or older, 70 years old or older, 80 years old or older, and sometimes no older than 100 years old, such as 90 years old, i.e., between the ages of about 50 and 100, e.g., 50, 55, 60, 65, 70, 75, 80, 85 or about 90 years old. The individual may suffer from an aging associated condition, e.g., cognitive impairment, associated with the natural aging process, e.g., M.C.I. Alternatively, the individual may be 50 years old or older, e.g., 60 years old or older, 70 years old or older, 80 years old or older, 90 years old or older, and sometimes no older than 100 years old, i.e., between the ages of about 50 and 100, e.g., 50, 55, 60, 65, 70, 75, 80, 85, 90, 95 or about 100 years old, and has not yet begun to show symptoms of an aging associated condition, e.g., cognitive impairment. In yet other embodiments, the individual may be of any age where the individual is suffering from a cognitive impairment due to an aging-associated disease, e.g., Alzheimer's disease, Parkinson's disease, frontotemporal dementia, Huntington's disease, amyotrophic lateral sclerosis, multiple sclerosis, glaucoma, myotonic dystrophy, dementia, and the like. In some instances, the individual is an individual of any age that has been diagnosed with an aging-associated disease that is typically accompanied by cognitive impairment, e.g., Alzheimer's disease, Parkinson's disease, frontotemporal dementia, progressive supranuclear palsy, Huntington's disease, amyotrophic lateral sclerosis, spinal muscular atrophy, multiple sclerosis, multi-system atrophy, glaucoma, ataxias, myotonic dystrophy, dementia, and the like, where the individual has not yet begun to show symptoms of cognitive impairment.

In those embodiments where an active agent is administered to the adult mammal, the active agent(s) may be administered to the adult mammal using any convenient administration protocol capable of resulting in the desired activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intravenous, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to the adult mammal, the amount or dosage is effective when administered for a suitable period of time, such as one week or longer, including two weeks or longer, such as 3 weeks or longer, 4 weeks or longer, 8 weeks or longer, etc., so as to evidence a reduction in the impairment, e.g., cognition decline and/or cognitive improvement in the adult mammal. For example, an effective dose is the dose that, when administered for a suitable period of time, such as at least about one week, and maybe about two weeks, or more, up to a period of about 3 weeks, 4 weeks, 8 weeks, or longer, will slow e.g., by about 20% or more, e.g., by 30% or more, by 40% or more, or by 50% or more, in some instances by 60% or more, by 70% or more, by 80% or more, or by 90% or more, e.g., or will halt, cognitive decline in a patient suffering from natural aging or an aging-associated disorder. In some instances, an effective amount or dose of active agent will not only slow or halt the progression of the disease condition but will also induce the reversal of the condition, i.e., will cause an improvement in cognitive ability. For example, in some instances, an effective amount is the amount that when administered for a suitable period of time, usually at least about one week, and maybe about two weeks, or more, up to a period of about 3 weeks, 4 weeks, 8 weeks, or longer will improve the cognitive abilities of an individual suffering from an aging associated cognitive impairment by, for example 1.5-fold, 2-fold, 3-fold, 4-fold, 5-fold, in some instances 6-fold, 7-fold, 8-fold, 9-fold, or 10-fold or more relative to cognition prior to administration of the blood product.

Where desired, effectiveness of treatment may be assessed using any convenient protocol. Cognition tests and IQ test for measuring cognitive ability, e.g., attention and concentration, the ability to learn complex tasks and concepts, memory, information processing, visuospatial function, the ability to produce and understanding language, the ability to solve problems and make decisions, and the ability to perform executive functions, are well known in the art, any of which may be used to measure the cognitive ability of the individual before and/or during and after treatment with the subject blood product, e.g., to confirm that an effective amount has been administered. These include, for example, the General Practitioner Assessment of Cognition (GPCOG) test, the Memory Impairment Screen, the Mini Mental State Examination (MMSE), the California Verbal Learning Test, Second Edition, Short Form, for memory, the Delis-Kaplan Executive Functioning System test, the Alzheimer's Disease Assessment Scale (ADAS-Cog), the Psychogeriatric Assessment Scale (PAS) and the like. Progression of functional brain improvements may be detected by brain imaging techniques, such as Magnetic Resonance Imaging (MRI) or Positron Emission Tomography (PET) and the like. A wide range of additional functional assessments may be applied to monitor activities of daily living, executive functions, mobility, etc. In some embodiments, the method comprises the step of measuring cognitive ability, and detecting a decreased rate of cognitive decline, a stabilization of cognitive ability, and/or an increase in cognitive ability after administration of the blood product as compared to the cognitive ability of the individual before the blood product was administered. Such measurements may be made a week or more after administration of the blood product, e.g., 1 week, 2 weeks, 3 weeks, or more, for instance, 4 weeks, 6 weeks, or 8 weeks or more, e.g., 3 months, 4 months, 5 months, or 6 months or more.

Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, the reduction in synaptic plasticity and loss of synapses that occurs during the natural aging process or during the progression of an aging-associated disorder. In other words, cells present in adult mammals treated in accordance with methods of the invention will become more responsive to cues, e.g., activity cues, which promote the formation and maintenance of synapses.

Performance of methods of the invention, e.g., as described above, may manifest as improvements in observed synaptic plasticity, both in vitro and in vivo as an induction of long-term potentiation. For example, the induction of LTP in neural circuits may be observed in awake individuals, e.g., by performing non-invasive stimulation techniques on awake individuals to induce LTP-like long-lasting changes in localized neural activity (Cooke S F, Bliss T V (2006) Plasticity in the human central nervous system. Brain. 129(Pt 7):1659-73); mapping plasticity and increased neural circuit activity in individuals, e.g., by using positron emission tomography, functional magnetic resonance imaging, and/or transcranial magnetic stimulation (Cramer and Bastings, “Mapping clinically relevant plasticity after stroke,” Neuropharmacology (2000)39:842-51); and by detecting neural plasticity following learning, i.e., improvements in memory, e.g., by assaying retrieval-related brain activity (Buchmann et al., “Prion protein M129V polymorphism affects retrieval-related brain activity,” Neuropsychologia. (2008) 46:2389-402) or, e.g., by imaging brain tissue by functional magnetic resonance imaging (fMRI) following repetition priming with familiar and unfamiliar objects (Soldan et al., “Global familiarity of visual stimuli affects repetition-related neural plasticity but not repetition priming,” Neuroimage. (2008) 39:515-26; Soldan et al., “Aging does not affect brain patterns of repetition effects associated with perceptual priming of novel objects,” J. Cogn. Neurosci. (2008) 20:1762-76). In some embodiments, the method includes the step of measuring synaptic plasticity, and detecting a decreased rate of loss of synaptic plasticity, a stabilization of synaptic plasticity, and/or an increase in synaptic plasticity after administration of the blood product as compared to the synaptic plasticity of the individual before the blood product was administered. Such measurements may be made a week or more after administration of the blood product, e.g., 1 week, 2 weeks, 3 weeks, or more, for instance, 4 weeks, 6 weeks, or 8 weeks or more, e.g., 3 months, 4 months, 5 months, or 6 months or more.

In some instances, the methods result in a change in expression levels of one or more genes in one or more tissues of the host, e.g., as compared to a suitable control (such as described in the Experimental section, below). The change in expression level of a given gene may be 0.5-fold or greater, such as 1.0-fold or greater, including 1.5-fold or greater. The tissue may vary, and in some instances is nervous system tissue, e.g., central nervous system tissue, including brain tissue, e.g., hippocampal tissue. In some instances, the modulation of hippocampal gene expression is manifested as enhanced hippocampal plasticity, e.g., as compared to a suitable control.

In some instances, treatment results in an enhancement in the levels of one or more proteins in one or more tissues of the host, e.g., as compared to a suitable control (such as described in the Experimental section, below). The change in protein level of a given protein may be 0.5-fold or greater, such as 1.0 fold or greater, including 1.5 fold or greater, where in some instances the level may approach that of a healthy wild-type level, e.g., within 50% or less, such as 25% or less, including 10% or less, e.g., 5% or less of the healthy wild-type level. The tissue may vary, and in some instances is nervous system tissue, e.g., central nervous system tissue, including brain tissue, e.g., hippocampal tissue.

In some instances, the methods result in one or more structural changes in one or more tissues. The tissue may vary, and in some instances is nervous system tissue, e.g., central nervous system tissue, including brain tissue, e.g., hippocampal tissue. Structure changes of interest include an increase in dendritic spine density of mature neurons in the dentate gyrus (DG) of the hippocampus, e.g., as compared to a suitable control. In some instances, the modulation of hippocampal structure is manifested as enhanced synapse formation, e.g., as compared to a suitable control. In some instances, the methods may result in an enhancement of long-term potentiation, e.g., as compared to a suitable control. In some instance, the structure change of interest include an increase in synaptic number in the dentate gyrus or CA1 regions of the hippocampus.

In some instances, practice of the methods, e.g., as described above, results in an increase in neurogenesis in the adult mammal. The increase may be identified in a number of different ways, e.g., as described below in the Experimental section. In some instances, the increase in neurogenesis manifests as an increase the amount of Dcx-positive immature neurons, e.g., where the increase may be 2-fold or greater. In some instances, the increase in neurogenesis manifests as an increase in the number of BrdU/NeuN positive cells, where the increase may be 2-fold or greater.

In some instances, the methods result in enhancement in learning and memory, e.g., as compared to a suitable control. Enhancement in learning and memory may be evaluated in a number of different ways, e.g., the contextual fear conditioning and/or radial arm water maze (RAWM) paradigms described in the experimental section, below. When measured by contextual fear conditioning, treatment results in some instances in increased freezing in contextual, but not cued, memory testing. When measured by RAWM, treatment results in some instances in enhanced learning and memory for platform location during the testing phase of the task. In some instances, treatment is manifested as enhanced cognitive improvement in hippocampal-dependent learning and memory, e.g., as compared to a suitable control.

In some embodiments, administration of the TIMP2 recombinant proteins, e.g., as described above, may be performed in conjunction with an active agent having activity suitable to treat aging associated cognitive impairment. For example, a number of active agents have been shown to have some efficacy in treating the cognitive symptoms of Alzheimer's disease (e.g., memory loss, confusion, and problems with thinking and reasoning), e.g., cholinesterase inhibitors (e.g., Donepezil, Rivastigmine, Galantamine, Tacrine), Memantine, and Vitamin E. As another example, a number of agents have been shown to have some efficacy in treating behavioral or psychiatric symptoms of Alzheimer's Disease, e.g., citalopram (Celexa), fluoxetine (Prozac), paroxeine (Paxil), sertraline (Zoloft), trazodone (Desyrel), lorazepam (Ativan), oxazepam (Serax), aripiprazole (Abilify), clozapine (Clozaril), haloperidol (Haldol), olanzapine (Zyprexa), quetiapine (Seroquel), risperidone (Risperdal), and ziprasidone (Geodon).

In some aspects of the subject methods, the method further comprises the step of measuring cognition and/or synaptic plasticity after treatment, e.g., using the methods described herein or known in the art, and determining that the rate of cognitive decline or loss of synaptic plasticity have been reduced and/or that cognitive ability or synaptic plasticity have improved in the individual. In some such instances, the determination is made by comparing the results of the cognition or synaptic plasticity test to the results of the test performed on the same individual at an earlier time, e.g., 2 weeks earlier, 1 month earlier, 2 months earlier, 3 months earlier, 6 months earlier, 1 year earlier, 2 years earlier, 5 years earlier, or 10 years earlier, or more.

In some embodiments, the subject methods further include diagnosing an individual as having a cognitive impairment, e.g., using the methods described herein or known in the art for measuring cognition and synaptic plasticity, prior to administering the subject plasma comprising blood product. In some instances, the diagnosing will comprise measuring cognition and/or synaptic plasticity and comparing the results of the cognition or synaptic plasticity test to one or more references, e.g., a positive control and/or a negative control. For example, the reference may be the results of the test performed by one or more age matched individuals that experience aging-associated cognitive impairments (i.e., positive controls) or that do not experience aging-associated cognitive impairments (i.e., negative controls). As another example, the reference may be the results of the test performed by the same individual at an earlier time, e.g., 2 weeks earlier, 1 month earlier, 2 months earlier, 3 months earlier, 6 months earlier, 1 year earlier, 2 years earlier, 5 years earlier, or 10 years earlier, or more.

In some embodiments, the subject methods further comprise diagnosing an individual as having an aging-associated disorder, e.g., Alzheimer's disease, Parkinson's disease, frontotemporal dementia, progressive supranuclear palsy, Huntington's disease, amyotrophic lateral sclerosis, spinal muscular atrophy, multiple sclerosis, multi-system atrophy, glaucoma, ataxias, myotonic dystrophy, dementia, and the like. Methods for diagnosing such aging-associated disorders are well-known in the art, any of which may be used by the ordinarily skilled artisan in diagnosing the individual. In some embodiments, the subject methods further comprise both diagnosing an individual as having an aging associated disorder and as having a cognitive impairment.

c. Utility

The subject compositions and methods find use in treating, including preventing, aging-associated impairments and conditions associated therewith, such as impairments in the cognitive ability of individuals. Individuals suffering from or at risk of developing an aging-associated cognitive impairments include individuals that are about 50 years old or older, e.g., 60 years old or older, 70 years old or older, 80 years old or older, 90 years old or older, and usually no older than 100 years old, i.e., between the ages of about 50 and 100, e.g., 50, 55, 60, 65, 70, 75, 80, 85, 90, 95 or about 100 years old, and are suffering from cognitive impairment associated with natural aging process, e.g., mild cognitive impairment (M.C.I.); and individuals that are about 50 years old or older, e.g., 60 years old or older, 70 years old or older, 80 years old or older, 90 years old or older, and usually no older than 100 years old, i.e., between the ages of about 50 and 90, e.g., 50, 55, 60, 65, 70, 75, 80, 85, 90, 95 or about 100 years old, that have not yet begun to show symptoms of cognitive impairment. Examples of cognitive impairments that are due to natural aging include the following:
Mild cognitive impairment (M.C.I.) is a modest disruption of cognition that manifests as problems with memory or other mental functions such as planning, following instructions, or making decisions that have worsened over time while overall mental function and daily activities are not impaired. Thus, although significant neuronal death does not typically occur, neurons in the aging brain are vulnerable to sub-lethal age-related alterations in structure, synaptic integrity, and molecular processing at the synapse, all of which impair cognitive function.

Individuals suffering from or at risk of developing an aging-associated cognitive impairment that will benefit from treatment with the subject plasma-comprising blood product, e.g., by the methods disclosed herein, also include individuals of any age that are suffering from a cognitive impairment due to an aging-associated disorder; and individuals of any age that have been diagnosed with an aging-associated disorder that is typically accompanied by cognitive impairment, where the individual has not yet begun to present with symptoms of cognitive impairment. Examples of such aging-associated disorders include the following:
Alzheimer's disease (AD). Alzheimer's disease is a progressive, inexorable loss of cognitive function associated with an excessive number of senile plaques in the cerebral cortex and subcortical gray matter, which also contains b-amyloid and neurofibrillary tangles consisting of tau protein. The common form affects persons>60 yr. old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, synapses, and ultimately neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

Parkinson's Disease. Parkin son's Disease (PD) is an idiopathic, slowly progressive, degenerative CNS disorder characterized by slow and decreased movement, muscular rigidity, resting tremor, and postural instability. Originally considered primarily a motor disorder, PD is now recognized to also affect cognition, behavior, sleep, autonomic function, and sensory function. The most common cognitive impairments include an impairment in attention and concentration, working memory, executive function, producing language, and visuospatial function.

In primary Parkinson's disease, the pigmented neurons of the substantia nigra, locus caeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonism results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including striatonigral degeneration.

Frontotemporal dementia. Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. Symptoms are classified into three groups based on the functions of the frontal and temporal lobes affected: Behavioral variant FTD (bvFTD), with symptoms include lethargy and aspontaneity on the one hand, and disinhibition on the other; progressive nonfluent aphasia (PNFA), in which a breakdown in speech fluency due to articulation difficulty, phonological and/or syntactic errors is observed but word comprehension is preserved; and semantic dementia (SD), in which patients remain fluent with normal phonology and syntax but have increasing difficulty with naming and word comprehension. Other cognitive symptoms common to all FTD patients include an impairment in executive function and ability to focus. Other cognitive abilities, including perception, spatial skills, memory and praxis typically remain intact. FTD can be diagnosed by observation of reveal frontal lobe and/or anterior temporal lobe atrophy in structural MRI scans.

A number of forms of FTD exist, any of which may be treated or prevented using the subject methods and compositions. For example, one form of frontotemporal dementia is Semantic Dementia (SD). SD is characterized by a loss of semantic memory in both the verbal and non-verbal domains. SD patients often present with the complaint of word-finding difficulties. Clinical signs include fluent aphasia, anomia, impaired comprehension of word meaning, and associative visual agnosia (the inability to match semantically related pictures or objects). As the disease progresses, behavioral and personality changes are often seen similar to those seen in frontotemporal dementia although cases have been described of ‘pure’ semantic dementia with few late behavioral symptoms. Structural MRI imaging shows a characteristic pattern of atrophy in the temporal lobes (predominantly on the left), with inferior greater than superior involvement and anterior temporal lobe atrophy greater than posterior.

As another example, another form of frontotemporal dementia is Pick's disease (PiD, also PcD). A defining characteristic of the disease is build-up of tau proteins in neurons, accumulating into silver-staining, spherical aggregations known as “Pick bodies”. Symptoms include loss of speech (aphasia) and dementia. Patients with orbitofrontal dysfunction can become aggressive and socially inappropriate. They may steal or demonstrate obsessive or repetitive stereotyped behaviors. Patients with dorsomedial or dorsolateral frontal dysfunction may demonstrate a lack of concern, apathy, or decreased spontaneity. Patients can demonstrate an absence of self-monitoring, abnormal self-awareness, and an inability to appreciate meaning. Patients with gray matter loss in the bilateral posterolateral orbitofrontal cortex and right anterior insula may demonstrate changes in eating behaviors, such as a pathologic sweet tooth. Patients with more focal gray matter loss in the anterolateral orbitofrontal cortex may develop hyperphagia. While some of the symptoms can initially be alleviated, the disease progresses, and patients often die within two to ten years.

Huntington's disease. Huntington's disease (HD) is a hereditary progressive neurodegenerative disorder characterized by the development of emotional, behavioral, and psychiatric abnormalities; loss of intellectual or cognitive functioning; and movement abnormalities (motor disturbances). The classic signs of HD include the development of chorea—involuntary, rapid, irregular, jerky movements that may affect the face, arms, legs, or trunk—as well as cognitive decline including the gradual loss of thought processing and acquired intellectual abilities. There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.

Amyotrophic lateral sclerosis. Amyotrophic lateral sclerosis (ALS) is a rapidly progressive, invariably fatal neurological disease that attacks motor neurons. Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 years; 50% die within 3 years of onset, 20% live 5 years, and 10% live 10 years. Diagnostic features include onset during middle or late adult life and progressive, generalized motor involvement without sensory abnormalities. Nerve conduction velocities are normal until late in the disease. Recent studies have documented the presentation of cognitive impairments as well, particularly a reduction in immediate verbal memory, visual memory, language, and executive function.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Promoting the formation or new synapses or preventing synapse loss may maintain neuron function in these patients.

Multiple Sclerosis. Multiple Sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Common cognitive impairments include impairments in memory (acquiring, retaining, and retrieving new information), attention and concentration (particularly divided attention), information processing, executive functions, visuospatial functions, and verbal fluency. Common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.

Glaucoma. Glaucoma is a common neurodegenerative disease that affects retinal ganglion cells (RGCs). Evidence supports the existence of compartmentalized degeneration programs in synapses and dendrites, including in RGCs. Recent evidence also indicates a correlation between cognitive impairment in older adults and glaucoma (Yochim B P, et al. Prevalence of cognitive impairment, depression, and anxiety symptoms among older adults with glaucoma. J Glaucoma. 2012; 21(4):250-254).

Myotonic dystrophy. Myotonic dystrophy (D M) is an autosomal dominant multisystem disorder characterized by dystrophic muscle weakness and myotonia. The molecular defect is an expanded trinucleotide (CTG) repeat in the 3′ untranslated region of the myotonin-protein kinase gene on chromosome 19q. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities (e.g., diabetes mellitus). Mental retardation is common in severe congenital forms, while an aging-related decline of frontal and temporal cognitive functions, particularly language and executive functions, is observed in milder adult forms of the disorder. Severely affected persons die by their early 50s.

Dementia. Dementia describes class of disorders having symptoms affecting thinking and social abilities severely enough to interfere with daily functioning. Other instances of dementia in addition to the dementia observed in later stages of the aging associated disorders discussed above include vascular dementia, and dementia with Lewy bodies, described below.

In vascular dementia, or “multi-infarct dementia”, cognitive impairment is caused by problems in supply of blood to the brain, typically by a series of minor strokes, or sometimes, one large stroke preceded or followed by other smaller strokes. Vascular lesions can be the result of diffuse cerebrovascular disease, such as small vessel disease, or focal lesions, or both. Patients suffering from vascular dementia present with cognitive impairment, acutely or subacutely, after an acute cerebrovascular event, after which progressive cognitive decline is observed. Cognitive impairments are similar to those observed in Alzheimer's disease, including impairments in language, memory, complex visual processing, or executive function, although the related changes in the brain are not due to AD pathology but to chronic reduced blood flow in the brain, eventually resulting in dementia. Single photon emission computed tomography (SPECT) and positron emission tomography (PET) neuroimaging may be used to confirm a diagnosis of multi-infarct dementia in conjunction with evaluations involving mental status examination.

Dementia with Lewy bodies (DLB, also known under a variety of other names including Lewy body dementia, diffuse Lewy body disease, cortical Lewy body disease, and senile dementia of Lewy type) is a type of dementia characterized anatomically by the presence of Lewy bodies (clumps of alpha-synuclein and ubiquitin protein) in neurons, detectable in post mortem brain histology. Its primary feature is cognitive decline, particularly of executive functioning. Alertness and short-term memory will rise and fall. Persistent or recurring visual hallucinations with vivid and detailed pictures are often an early diagnostic symptom. DLB it is often confused in its early stages with Alzheimer's disease and/or vascular dementia, although, where Alzheimer's disease usually begins quite gradually, DLB often has a rapid or acute onset. DLB symptoms also include motor symptoms similar to those of Parkinson's. DLB is distinguished from the dementia that sometimes occurs in Parkinson's disease by the time frame in which dementia symptoms appear relative to Parkinson symptoms. Parkinson's disease with dementia (PDD) would be the diagnosis when dementia onset is more than a year after the onset of Parkinson's. DLB is diagnosed when cognitive symptoms begin at the same time or within a year of Parkinson symptoms.

Progressive supranuclear palsy. Progressive supranuclear palsy (PSP) is a brain disorder that causes serious and progressive problems with control of gait and balance, along with complex eye movement and thinking problems. One of the classic signs of the disease is an inability to aim the eyes properly, which occurs because of lesions in the area of the brain that coordinates eye movements. Some individuals describe this effect as a blurring. Affected individuals often show alterations of mood and behavior, including depression and apathy as well as progressive mild dementia. The disorder's long name indicates that the disease begins slowly and continues to get worse (progressive), and causes weakness (palsy) by damaging certain parts of the brain above pea-sized structures called nuclei that control eye movements (supranuclear). PSP was first described as a distinct disorder in 1964, when three scientists published a paper that distinguished the condition from Parkinson's disease. It is sometimes referred to as Steele-Richardson-Olszewski syndrome, reflecting the combined names of the scientists who defined the disorder. Although PSP gets progressively worse, no one dies from PSP itself.

Ataxia. People with ataxia have problems with coordination because parts of the nervous system that control movement and balance are affected. Ataxia may affect the fingers, hands, arms, legs, body, speech, and eye movements. The word ataxia is often used to describe a symptom of incoordination which can be associated with infections, injuries, other diseases, or degenerative changes in the central nervous system. Ataxia is also used to denote a group of specific degenerative diseases of the nervous system called the hereditary and sporadic ataxias which are the National Ataxia Foundation's primary emphases.

Multiple-system atrophy. Multiple-system atrophy (MSA) is a degenerative neurological disorder. MSA is associated with the degeneration of nerve cells in specific areas of the brain. This cell degeneration causes problems with movement, balance, and other autonomic functions of the body such as bladder control or blood-pressure regulation. The cause of MSA is unknown and no specific risk factors have been identified. Around 55% of cases occur in men, with typical age of onset in the late 50s to early 60s. MSA often presents with some of the same symptoms as Parkinson's disease. However, MSA patients generally show minimal if any response to the dopamine medications used for Parkinson's.

Frailty, Frailty Syndrome (“Frailty”) is a geriatric syndrome characterized by functional and physical decline including decreased mobility, muscle weakness, physical slowness, poor endurance, low physical activity, malnourishment, and involuntary weight loss. Such decline is often accompanied and a consequence of diseases such as cognitive dysfunction and cancer. However, Frailty can occur even without disease. Individuals suffering from Frailty have an increased risk of negative prognosis from fractures, accidental falls, disability, comorbidity, and premature mortality. (C. Buigues, et al. Effect of a Prebiotic Formulation on Frailty Syndrome: A Randomized, Double-Blind Clinical Trial, Int. J. Mol. Sci. 2016, 17, 932). Additionally, individuals suffering from Frailty have an increased incidence of higher health care expenditure. (Id.)

Common symptoms of Frailty can be determined by certain types of tests. For example, unintentional weight loss involves a loss of at least 10 lbs. or greater than 5% of body weight in the preceding year; muscle weakness can be determined by reduced grip strength in the lowest 20% at baseline (adjusted for gender and BMI); physical slowness can be based on the time needed to walk a distance of 15 feet; poor endurance can be determined by the individual's self-reporting of exhaustion; and low physical activity can be measured using a standardized questionnaire. (Z. Palace et al., The Frailty Syndrome, Today's Geriatric Medicine 7(1), at 18 (2014)).

In some embodiments, the subject methods and compositions find use in slowing the progression of aging-associated cognitive impairment. In other words, cognitive abilities in the individual will decline more slowly following treatment by the disclosed methods than prior to or in the absence of treatment by the disclosed methods. In some such instances, the subject methods of treatment include measuring the progression of cognitive decline after treatment and determining that the progression of cognitive decline is reduced. In some such instances, the determination is made by comparing to a reference, e.g., the rate of cognitive decline in the individual prior to treatment, e.g., as determined by measuring cognition prior at two or more time points prior to administration of the subject blood product.

The subject methods and compositions also find use in stabilizing the cognitive abilities of an individual, e.g., an individual suffering from aging-associated cognitive decline or an individual at risk of suffering from aging-associated cognitive decline. For example, the individual may demonstrate some aging-associated cognitive impairment, and progression of cognitive impairment observed prior to treatment with the disclosed methods will be halted following treatment by the disclosed methods. As another example, the individual may be at risk for developing an aging-associated cognitive decline (e.g., the individual may be aged 50 years old or older or may have been diagnosed with an aging-associated disorder), and the cognitive abilities of the individual are substantially unchanged, i.e., no cognitive decline can be detected, following treatment by the disclosed methods as compared to prior to treatment with the disclosed methods.

The subject methods and compositions also find use in reducing cognitive impairment in an individual suffering from an aging-associated cognitive impairment. In other words, cognitive ability is improved in the individual following treatment by the subject methods. For example, the cognitive ability in the individual is increased, e.g., by 2-fold or more, 5-fold or more, 10-fold or more, 15-fold or more, 20-fold or more, 30-fold or more, or 40-fold or more, including 50-fold or more, 60-fold or more, 70-fold or more, 80-fold or more, 90-fold or more, or 100-fold or more, following treatment by the subject methods relative to the cognitive ability that is observed in the individual prior to treatment by the subject methods. In some instances, treatment by the subject methods and compositions restores the cognitive ability in the individual suffering from aging-associated cognitive decline, e.g., to their level when the individual was about 40 years old or less. In other words, cognitive impairment is abrogated.

d. Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of treating cognitive indications with the various TIMP2 recombinant protein constructs.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a Website address which may be used via the internet to access the information at a removed site. Any convenient means may be present in the kits.

### 6. EXAMPLES

The following examples are provided by way of illustration and not by way of limitation.

### A. Experimental Examples

i. Recombinant Protein Constructs

1. Purification of TIMP2 Recombinant Protein Constructs

The different TIMP2 proteins were purified either using anion exchange chromatography (AIEX), immobilized metal affinity chromatography (IMAC) with His tagged proteins, or protein A chromatography (ProA) with Fc fusions. These compositions were produced by human embryotic kidney (HEK) cells transiently transfected. Different cell lines (e.g., CHO) could be used, or stable cell lines could be developed. The proteins were purified from the cell culture supernatant (CCS) after centrifugal clarification of the producing cells and subsequent filtration (which is optional) of the CCS. Proteins were purified by IMAC, ALEX, or ProA with an optional SEC polishing step, followed by concentration and buffer exchange to phosphate buffered saline (PBS). All samples were tested for endotoxin (<5 EU/mL) prior to proceeding with in vivo studies.

a. Human IL-2 Signal Peptide

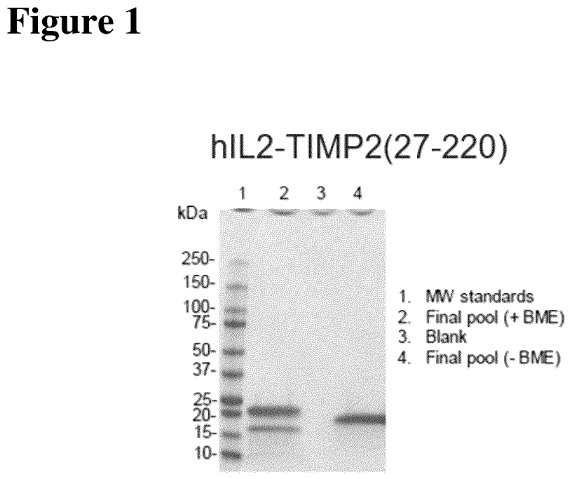

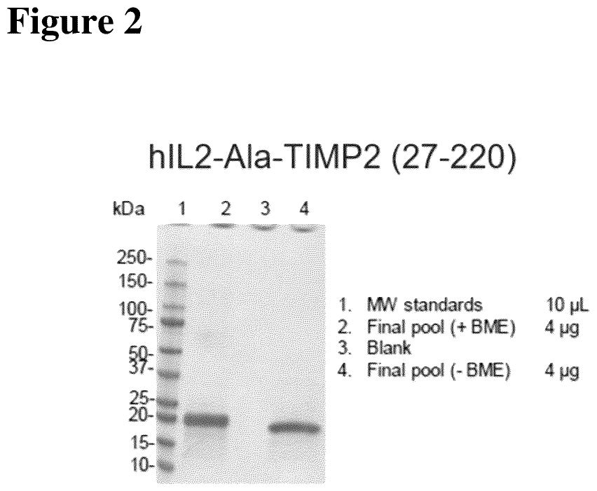

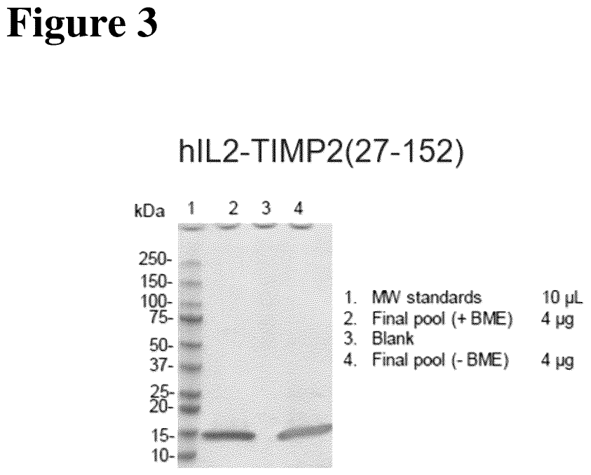

Several TIMP2 recombinant construct proteins employing the human interleukin 2 (IL-2) signal peptide sequence in place of the native TIMP2 signal peptide were purified. FIG. 1, FIG. 2, and FIG. 3 display SDS-PAGE images of purified TIMP2 proteins using the signal peptide from human IL-2 (SEQ ID NO: 23). These construct proteins include hIL2-TIMP2(27-220) (SEQ ID NO: 1), hIL2-Ala-TIMP2(27-220) (SEQ ID NO: 3), and HIL2-TIMP2(27-152) (SEQ ID NO: 2), respectively.

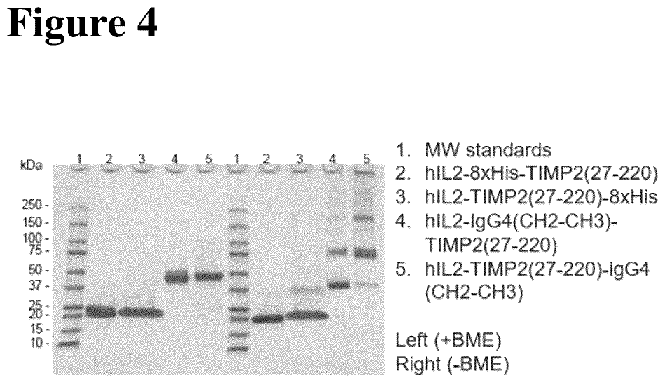

FIG. 4 displays an SDS-PAGE image of purified hIL2-8×His-TIMP2(27-220) (SEQ ID NO: 5, Column/well 2), hIL2-TIMP2(27-220)-8×His (SEQ ID NO:6, Column/well 3), hIL2-IgG4(CH2-CH3)-TIMP2(27-220) (SEQ ID NO: 7, Column/well 4), and hIL2-TIMP2(27-220)-IgG4(CH2-CH3) (SEQ ID NO: 8, Column/well 5). Left-handed columns/wells 1-5 contained beta-mercaptoethanol (BME+) and right-handed columns/wells 1-5 were absent beta-mercaptoethanol (BME−) in the sample buffer.

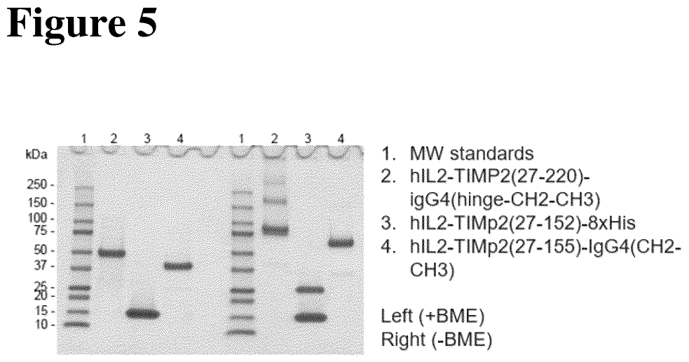

FIG. 5 displays an SDS-PAGE image of purified hIL2-TIMP2(27-220)-IgG4(hinge-CH2-CH3) (SEQ ID NO: 9, Column/well 2), hIL2-TIMP2(27-152)-8×His (SEQ ID NO: 10, Column/well 3), and hIL2-TIMP2(27-155)-IgG4(CH2-CH3) (SEQ ID NO: 12, Column/well 4). Left-handed columns/wells 1˜4 contained beta-mercaptoethanol (BME+) and right-handed columns/wells 1˜4 were absent beta-mercaptoethanol (BME−) in the sample buffer.

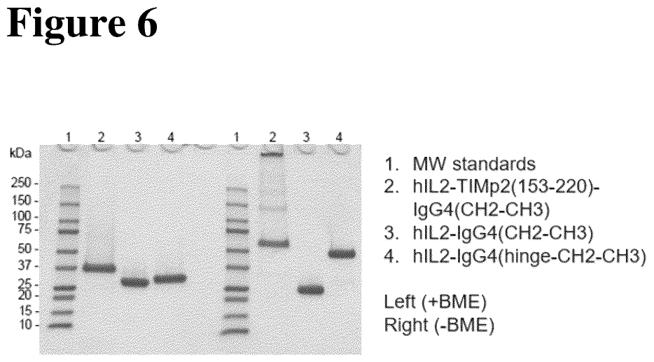

FIG. 6 displays an SDS-PAGE image of purified hIL2-TIMP2(153-220)-IgG4(hinge-CH2-CH3) (SEQ ID NO: 13, Column/well 2), hIL2-IgG4 (SEQ ID NO: 14, Column/well 3), and hIL2-IgG4(hinge-CH2-CH3) (SEQ ID NO: 15, Column/well 4). Left-handed columns/wells 1˜4 contained beta-mercaptoethanol (BME+) and right-handed columns/wells 1˜4 were absent beta-mercaptoethanol (BME−) in the sample buffer.

b. Endogenous Human TIMP2 Signal Peptide

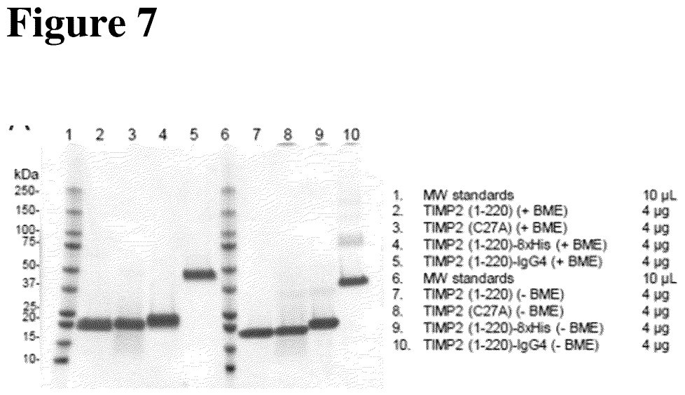

FIG. 7 displays an SDS-PAGE image of purified TIMP2 proteins using the endogenous human TIMP2 signal peptide (SEQ ID NO: 24). These recombinant protein constructs include TIMP2(1-220) (SEQ ID NO: 17, Columns/wells 1 and 7), TIMP2(C27A) (SEQ ID NO: 18, Columns 2 and 8), TIMP2(1-220)-8×His (SEQ ID NO: 19, Columns/wells 3 and 9), and TIMP2(1-220)-IgG4 (SEQ ID NO: 20, Columns/wells 5 and 10). Columns 1-5 contained beta-mercaptoethanol (BME+) and right-handed columns 1-5 were absent beta=mercaptoethanol (BME−).

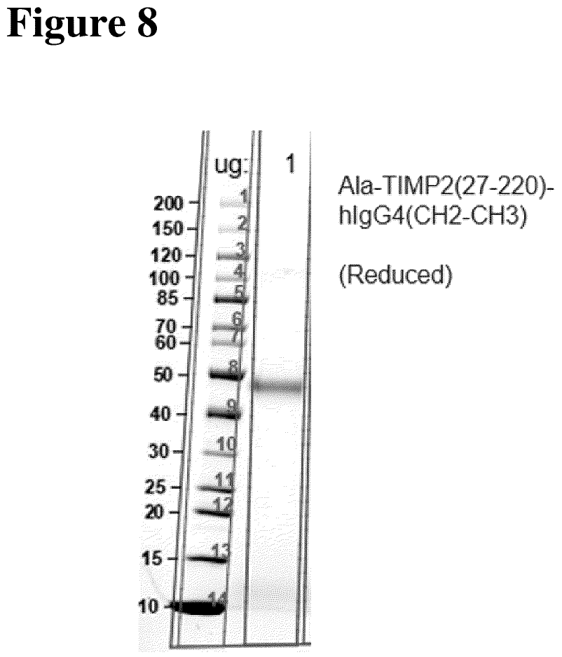

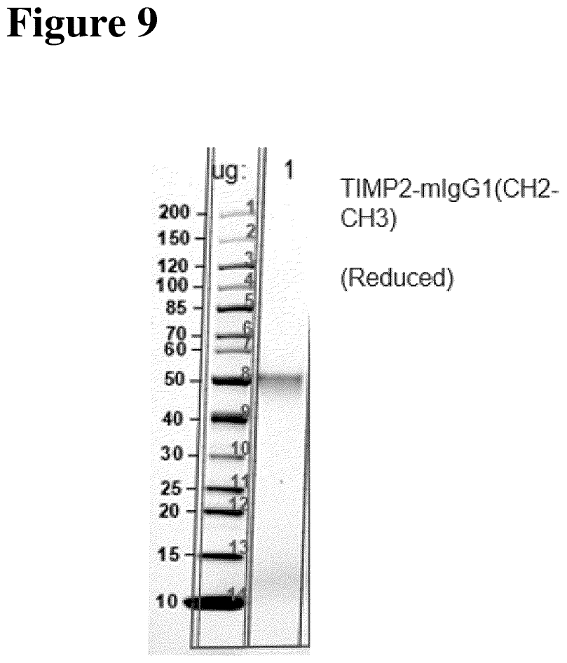

FIG. 8 displays an SDS-PAGE image of purified and reduced Ala-TIMP2(27-220)-hIgG4(CH2-CH3) (SEQ ID NO: 34). FIG. 9 displays an SDS-PAGE image of purified and reduced TIMP2-mIgG1(CH2-CH3) (SEQ ID NO: 22).

2. Characterization of TIMP2 Recombinant Protein Constructs

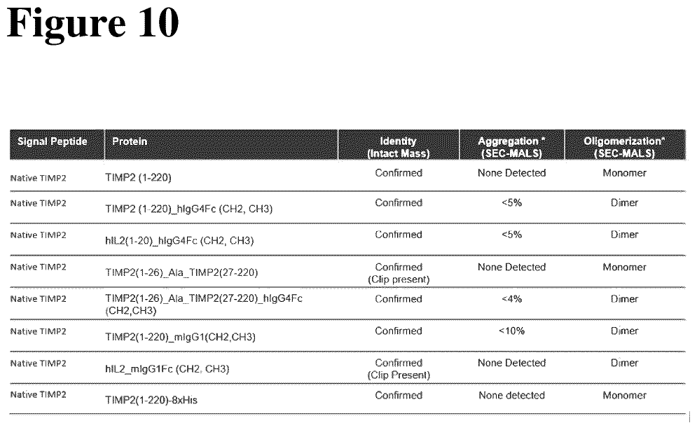

FIG. 10 is a table characterizing data for different lots of TIMP2 proteins with endogenous signal peptide (SEQ ID NO: 24). Row 1 shows characterization of SEQ ID NO: 16. Row 2 shows characterization of SEQ ID NO: 35. Row 3 shows characterization of SEQ ID NO: 35. Row 4 shows characterization of SEQ ID NO: 36. Row 5 shows characterization of SEQ ID NO: 37. Row 6 shows characterization of SEQ ID NO: 22. Row 7 shows characterization of SEQ ID NO: 38. Row 8 shows characterization of SEQ ID NO: 19.

TIMP2 proteins were shown to be tolerant of freeze/thaw treatment. The table in Figure summarizes data from LC/MS intact mass characterization and SEC-MALS to evaluate aggregation state and oligomerization state.

3. Binding of Constructs to Matrix Metalloproteases (MMPs)

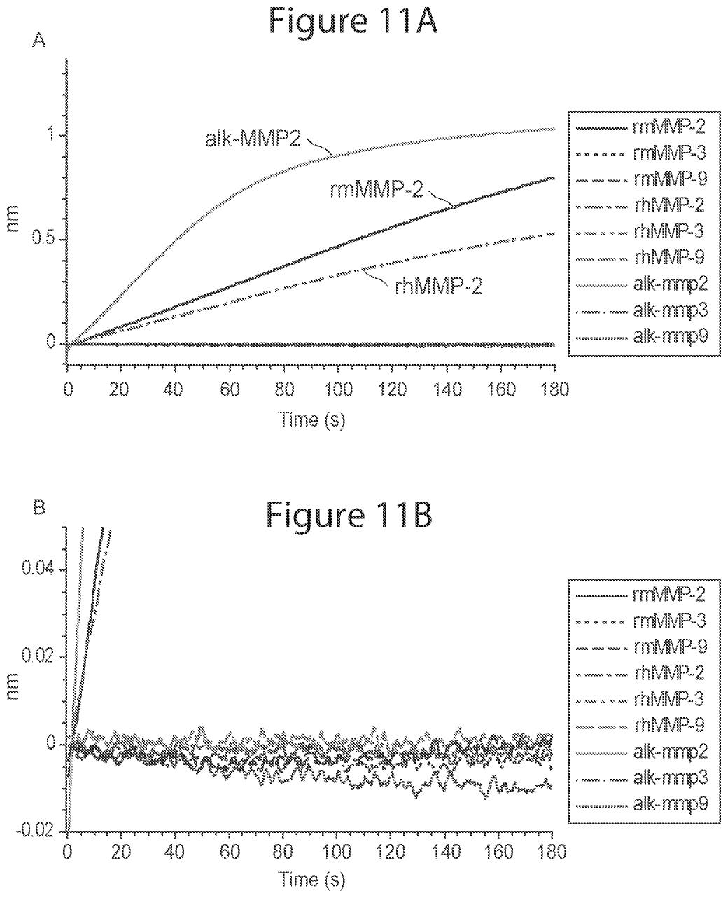

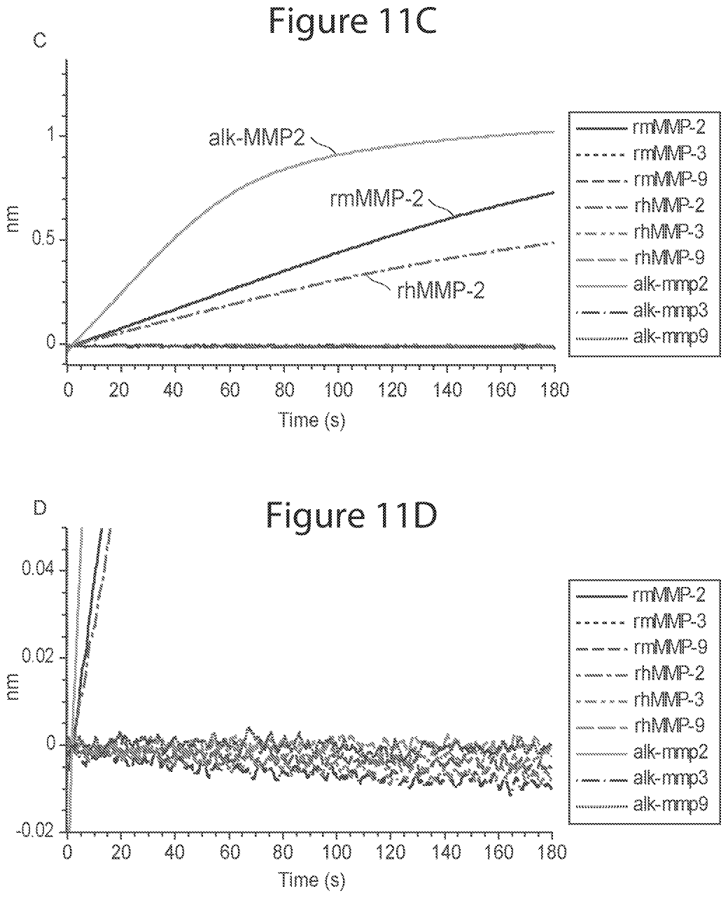

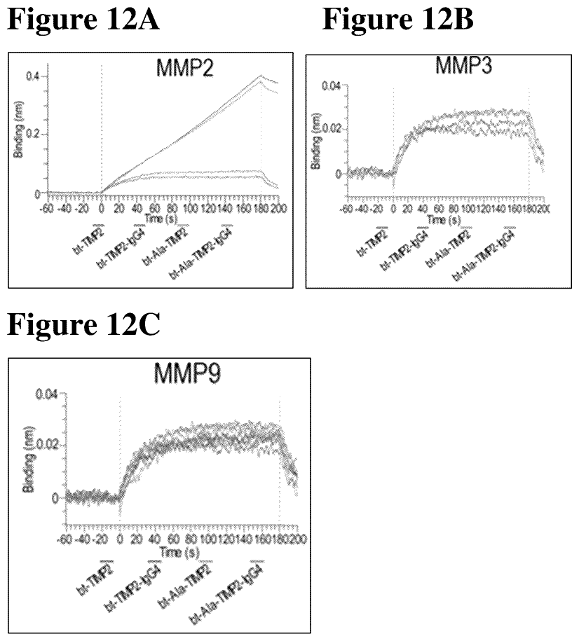

Binding interactions of TIMP2 recombinant protein constructs with MMP proteins (commercially sourced) were evaluated. Binding was evaluated by either BLI or SPR. FIGS. 11A, 11B, 11C, and 11D, as well as FIGS. 12A, 12B, and 12C show BLI studies performed on the Octet Red96e. TIMP2 and Ala-TIMPI2-hIgGr were captured using ProA Octet tips or streptavidin Octet tips depending on biotinylation state. MMPs (22.5 nM) were associated at captured biotinylated proteins. Recombinant MMPs were sourced mouse and human. The Alk MMPs are human MMPs provided for cross comparison to kits utilized at another, additional laboratory. All curves were reference subtracted. FIG. 11A shows that biotinylated TIMP2 (45 nM) was captured by streptavidin Octet tips. FIG. 11B is a zoomed-in view of the chromatogram in FIG. 11A depicting observed binding curves of MMP3 and MMP9. FIG. 11C shows that biotinylated Ala-TIMP2 (45 nM) was captured by streptavidin Octet tips. FIG. 11D is a zoomed-in view of the chromatogram in FIG. 11C depicting observed binding curves of MMP3 and MMP9. FIG. 12A shows that biotinylated TIMP2 (45 nM) was captured by streptavidin Octet tips showing association with MMP2. FIG. 12B shows that biotinylated TIMP2 (45 nM) was captured by streptavidin Octet tips showing association with MMP3. FIG. 12C shows that biotinylated TIMP2 (45 nM) was captured by streptavidin Octet tips showing association with MMP9. Ala-TIMP2 (45 nM) was captured by streptavidin Octet tips. Thus, binding of each TIMP2 construct to each MMP was confirmed.

4. Binding of TIMP2 Constructs to α3β1 Integrin

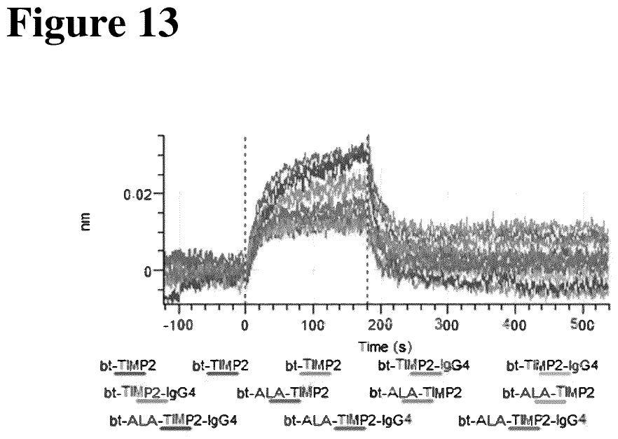

Binding interactions of TIMP2 recombinant protein constructs with α3β1 integrin were also evaluated (FIG. 13). TIMP2 and Ala-TIMP2 were biotinylated with a 1:1 molar ratio of biotin to protein. TIMP2-hIgG4 and Ala-TIMP2-hIgG4 were biotinylated with a 1:1 molar ratio of biotin to dimerized protein. α3β1 integrin was associated at 22.5 nM to captured biotinylated protein. Binding observed for α3β1 integrin. BLI studies were performed on the Octet Red96e. Biotinylated TIMP2, Ala-TIMP2, TIMP2-hIgG4, and Ala-TIMP2-hIgG4 dimer were captured by Streptavidin Octet Tips at 45 nM. All curves are reference-subtracted.

5. Binned Association for Different TIMP2 and MMP Pairings

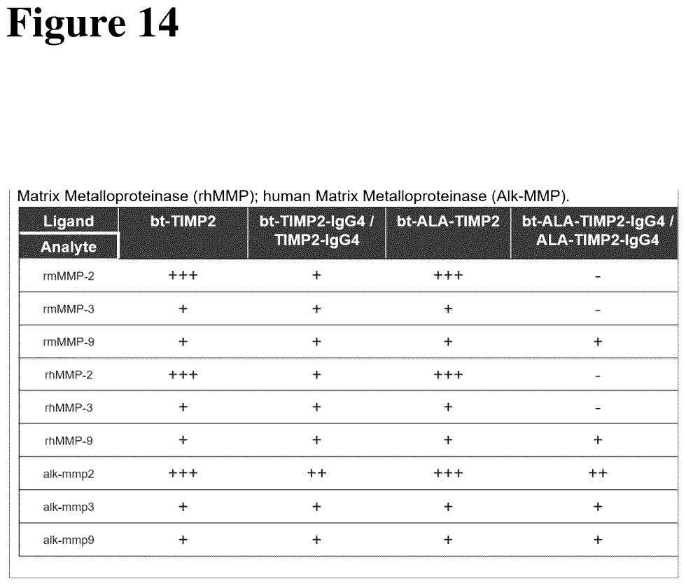

FIG. 14 is a summary table of association studies from BLI. “−” indicates no observable association; “+” indicates association corresponding to a nanometer shift between 0 and 0.05; “++” indicates an association corresponding to a nanometer shift between 0.05 and 0.2; “+++” indicates an association corresponding to a nanometer shift greater than 0.2. Association ranges were determined by visual inspection of naturally occurring groups. (Abbreviations: recombinant mouse Matrix Metalloproteinase (rmMMP); recombinant human Matrix Metalloproteinase (rhMMP); human Matrix Metalloproteinase (Alk-MMP)).

ii. In Vivo Experiments Using Recombinant Protein Constructs

1. Half-Life of IgG4 C-Terminal Fusion Protein

Aged C57BL/6 mice were injected with a single dose of vehicle or protein 250 μg/kg i.p. (human TIMP2 “TIMP2”, Ala-TIMP2, or Ala-TIMP2-hIgG4) then sacrificed at 6 hours, 24 hours, 48 hours, or 96 hours post-administration (n=1-3 per treatment group per time point). EDTA blood was collected by cardiac puncture then plasma was isolated. Human TIMP2 levels were detected by running a DuoSet TIMP2 ELISA (DY971, R&D Systems) on terminal plasma diluted 1:25 in duplicate.

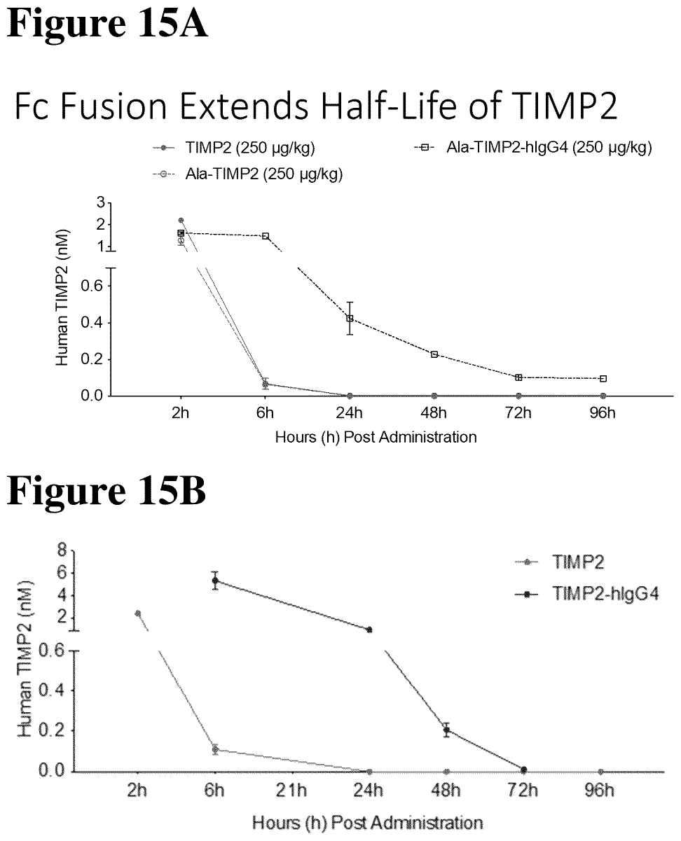

FIG. 15 shows that TIMP2 and Ala-TIMP2 were both detectable at 6 h but not 24 h. The human IgG4 fusion (Ala-TIMP2-hIgG4) extended the half-life such that the Ala-TIMP2-hIgG4 protein was detectable at 48 h. The Ala-TIMP2-hIgG4 protein was barely detectable after 72 h. Clearance of TIMP2 and Ala-TIMP were approximately 3×faster than TIMP2-hIgG4, Ala-TIMP2-hIgG4.

The hIgG4Fc fusion to the TIMP2 protein thus extended the half-life. Based on this data, it was determined that TIMP2 requires daily dosing while hIgG4 fusion proteins can be dosed once every 3 days.

2. Effect of TIMP2 Constructs on MMP Activity

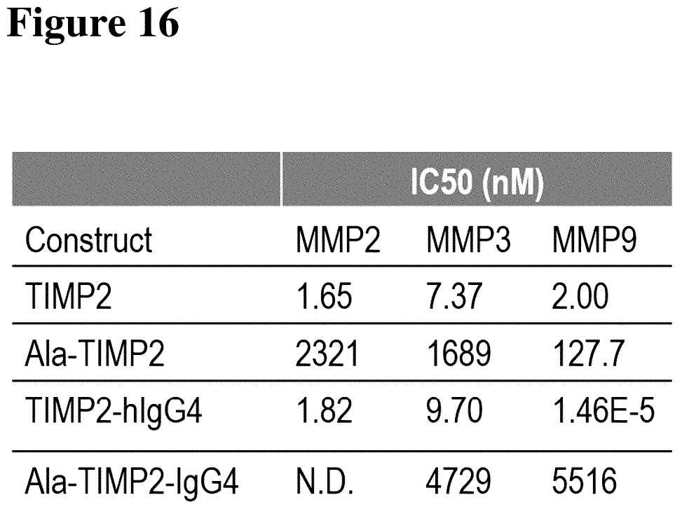

The ability of the TIMP2, Ala-TIMP2, TIMP2-hIgG4, and Ala-TIMP2-IgG4 recombinant protein constructs to inhibit MMPs was analyzed using three kits to different MMPs: SensoLyte 520 MMP-2 Assay Kit (AS-71151, AnaSpec), MMP-3 Assay Kit (AS-71152, AnaSpec), and MMP-9 Assay Kit (AS-71155, AnaSpec). The constructs were used at various known concentrations. After initiating the enzymatic reaction for a set amount of time (15 min for MMP2 and MMP3, 75 min for MMP9), the fluorescent intensity was measured using the BMG LABTECH CLARIOstar plate reader at 450 nm and wavelength correction set at 540 nm. Substrate only and substrate plus MMP2/3/9 controls were used to calculate the percent inhibition. The IC₅₀ for each MMP was calculated based on the known concentrations and measured inhibitory activity (see FIG. 16).

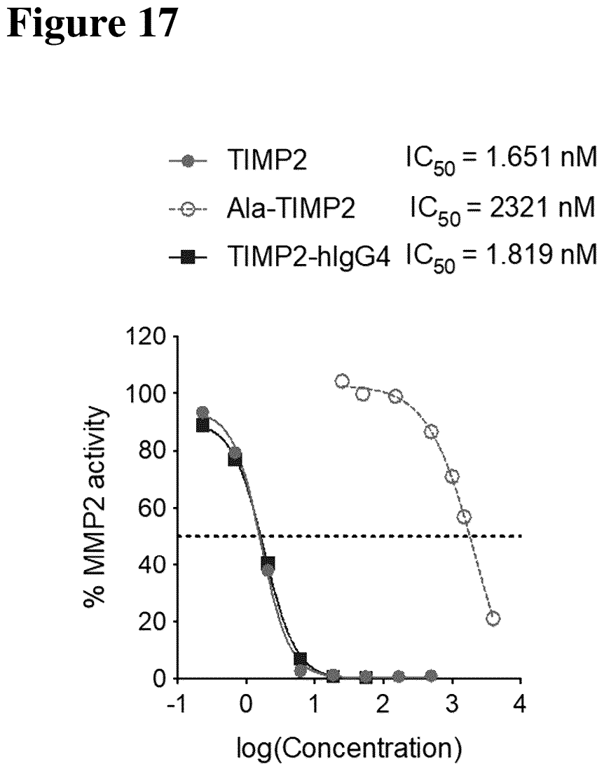

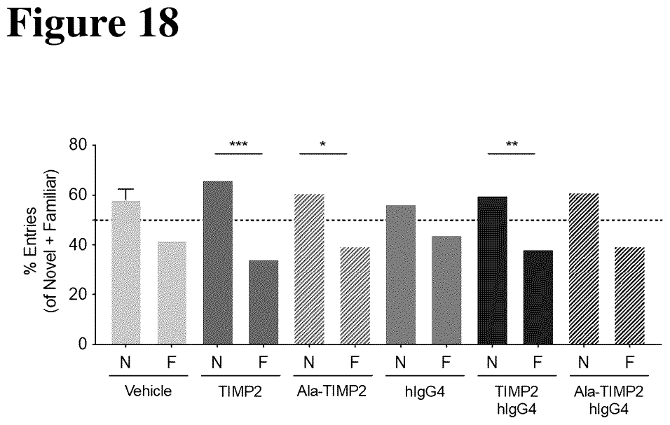

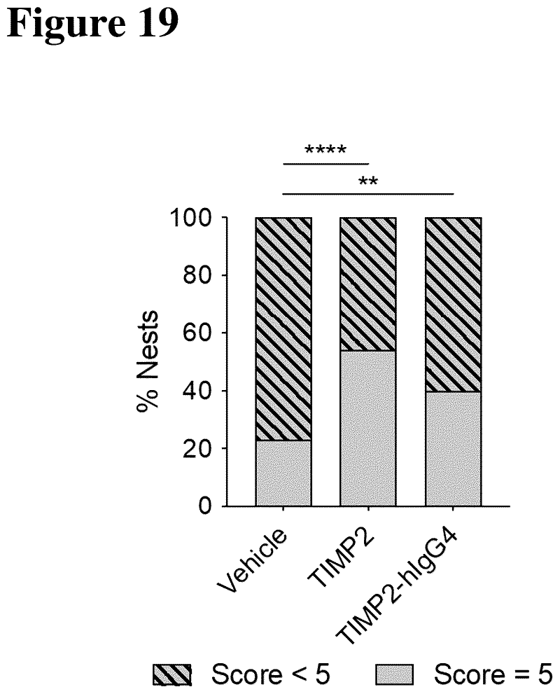

FIG. 17 shows that TIMP2 and TIMP2-hIgG4 performed similarly and inhibit MMP2 at biologically relevant levels. The inhibitory activity of Ala-TIMP2 was greatly reduced and below biologically relevant levels. The Ala-TIMP2-hIgG4 protein had even lower inhibitory activity, suggesting that the IgG4Fc itself may impact MMP inhibitory activity.

3. Effect of TIMP2 Constructs on Cognitive Function

a. Y-Maze

Aged C57BL/6 mice were treated for five weeks as follows: 1×PBS Vehicle 100 μL/dose i.p. daily; TIMP2(1-220), 250 μg/kg in 100 μL/dose i.p. daily; TIMP2(1-26)-Ala-TIMP2(27-220), 250 μg/kg in 100 μL/dose i.p. daily; TIMP2(1-220)-hIgG4Fc, 250 μg/kg in 100 μL/dose i.p. every third day and treated with vehicle on off days; TIMP2(1-26)-Ala-TIMP2(27-220)-hIgG4Fc, 250 μg/kg in 100 μL/dose i.p. every third day and treated with vehicle on off days; and hIgG4Fc, 250 μg/kg in 100 μL/dose i.p. every third day and treated with vehicle on off days. The mice were acclimated to the behavior room for at least 30 minutes prior to testing. Unique cues in the form of black shapes were adhered to the walls at the ends of two of the arms, while the third arm was un-cued and designated as the starting point for the mice. First, mice were individually placed in the starting arm and allowed to explore only one of the other two arms (familiar arm) for 5 minutes; the second arm (novel arm) was blocked off with an acrylic plastic wall identical to that of the rest of the apparatus. After 2.5 hours, each mouse was then returned to the maze with both arms now open to explore for 5 minutes. All movements were recorded and tracked for analysis using ANY-Maze Software. The number of entries into and the time spent in each of the two arms, familiar and novel, was measured. The total distance and velocity were also measured for the duration of the test. Percent entries were analyzed using a Wilcoxon Signed Rank Tests to compare the percent of novel entries for each group against 50%. N=13-16 per group.

Mice treated with TIMP2, Ala-TIMP2, and TIMP2-hIgG4 were able to remember the novel arm as measured by percent entries into the novel arm compared to 50% chance, but Vehicle, hIgG4 control, and Ala-TIMP2-hIgG4 could not distinguish between the arms. Hippocampal-dependent memory in the Y-maze therefore was improved with treatment of TIMP2, Ala-TIMP2, and TIMP2-hIgG4. Improvement in Y-maze performance with Ala-TIMP2 treatment also suggests that MMP inhibitory activity is not necessary for the cognitive improvement exhibited with TIMP2 treatment. This is surprising since TIMP2's role in inhibiting MMP function was expected to play an important part in TIMP's ability to improve cognitive performance associated with aged animals. Additionally, it has been shown previously that MMP levels increase with age and plays a detrimental role in cognitive performance. This suggests that abrogation of the MMP inhibitory activity of TIMP2 through such recombinant protein constructs can help to avoid unwanted side effects commonly reported with MMP inhibitors such as musculoskeletal pain and inflammation (see, e.g., Vandenbroucke R E, et al., Nature Rev. Drug Discov., 13:904-27 (2014) and Fingleton B, Semin Cell Dev Biol, 19(1):61-68 (2008)) resulting in pain and immobility in the shoulder joints, arthralgias, and contractures in the hands.

b. Nesting Behavior

Nesting scoring was based on a published protocol (Deacon R M J, Nature Protocols, 1(3), 1117-1119.). Mice were placed in a clean home cage and given two nestlets in the evening towards the end of their light cycle. The next morning (˜16 hours later), nests were scored by blinded experimenters on a scale of 1-5, with 5 being the most dome-like complete nest. Since most mice are proficient in building nests, mice were divided into two groups of either scores of 5 or scores below 5. Chi-squared tests were used to test statistical significance between groups.

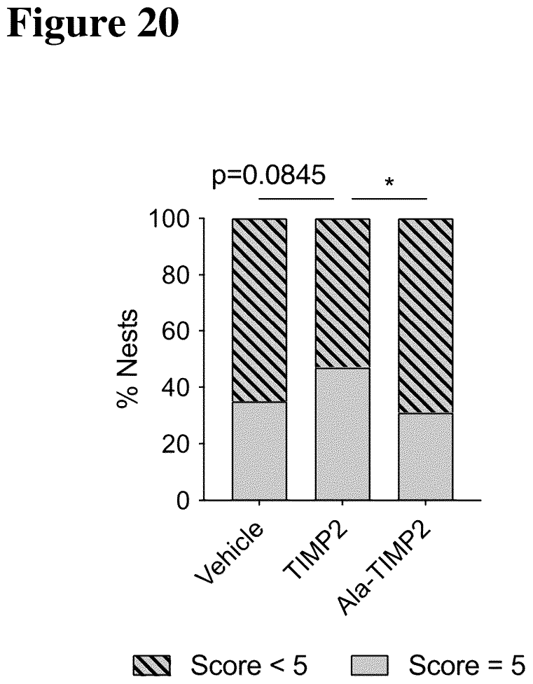

Both TIMP2 and TIMP2-hIgG4 improve nesting compared to vehicle-treated mice (N=10-13 per group); however, Ala-TIMP2 does not, suggesting that MMP inhibitory activity may be necessary for nesting improvements (N=13-17 per group) (see FIGS. 19 and 20).

4. Effect of TIMP2 Constructs on Excitatory Synaptic Density in Hippocampus

Aged C57BL/6 mice were treated for 25 days with 1×PBS Vehicle (100 μL/dose i.p. daily); TIMP2(1-220) (250 μg/kg in 100 μL/dose i.p. daily); TIMP2(1-220)-hIgG4Fc (250 μg/kg in 100 μL/dose i.p. every third day and treated with vehicle on off days); or TIMP2(1-26)-Ala-TIMP2(27-220) (250 μg/kg in 100 μL/dose i.p. daily). Mice were subsequently anesthetized with 2,2,2-tribromoethanol (Avertin, T48402-25G, Sigma Aldrich) and perfused with 0.9% saline transcardially. The brains were dissected and cut sagitally in two even halves. One half was fixed in 4% PFA (15714S, Electron Microscopy Sciences) in PBS for use in immunohistochemistry. After two days of fixation, the hemibrains were transferred to a 30% sucrose (S5-3, Fisher Scientific) in PBS solution and then changed again after one day. Hemibrains were sectioned coronally at 30 μm on a microtome at −22° C. Brain slices were collected sequentially into 12 tubes, so that every 12th section of the hippocampus was represented in a given tube. Brain sections were stored in cryoprotectant media composed of 30% ethylene glycol (E178-4, Fisher Scientific) and 30% glycerol (G5516, Sigma Aldrich) in a sodium phosphate solution at −20° C. until needed for staining. Sections were blocked in 10% goat serum with PBS and 1% triton for 1 hour. SYNAPSIN1/2 antibody (106 006, Synaptic Systems) at 1:1000 was used to stain the pre-synapse while Postsynaptic Density Protein-95 (PSD-95) antibody (3450S, Cell Signaling Technology) at 1:250 or Homer1 antibody (160 003, Synaptic Systems) at 1:500 was used to stain the excitatory post-synapse. Primary antibodies were incubated overnight at 4° C. in 3% goat serum in PBS with triton.

z-stack (0.18 μm step size) images in the CA1 and DG regions were acquired using a Zeiss LSM800 with Airyscan at 63×, Airyscan processed using Zen Blue 2.5 (Zeiss), and then quantified using the ImageJ macro SynapseCounter (https://github.com/SynPuCo/SynapseCounter) to measure pre-synaptic SYNAPSIN1/2 puncta; post-synaptic PSD-95 or Homer1 puncta; and juxtaposed signal for synapses. For the CA1 hippocampal region, synapses were analyzed from 6-8 ROIs from 10-18 mice per group using Kruskal-Wallis test followed by Dunn's multiple comparisons test (n=60-139 images from 10-18 mice per group). For the dentate gyrus (DG) hippocampal region, synapses were analyzed from 6-ROIs from 5-18 mice per group using Kruskal-Wallis test followed by Dunn's multiple comparisons test (n=42-142 images from 5-18 mice per group).

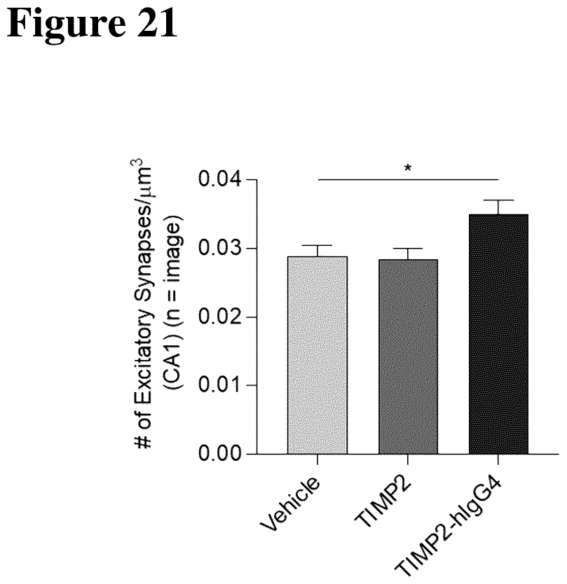

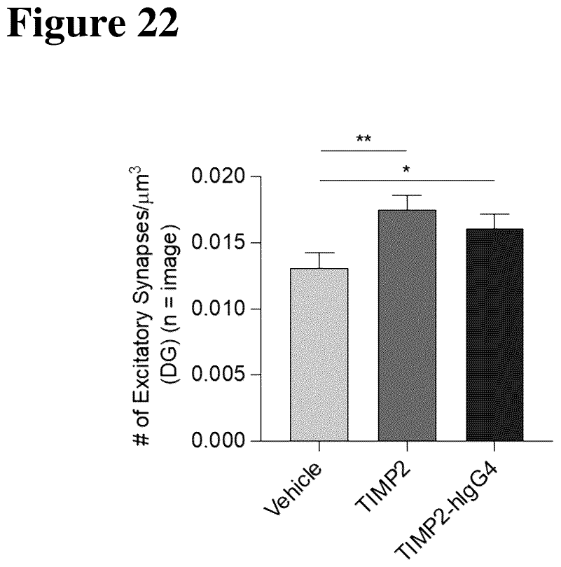

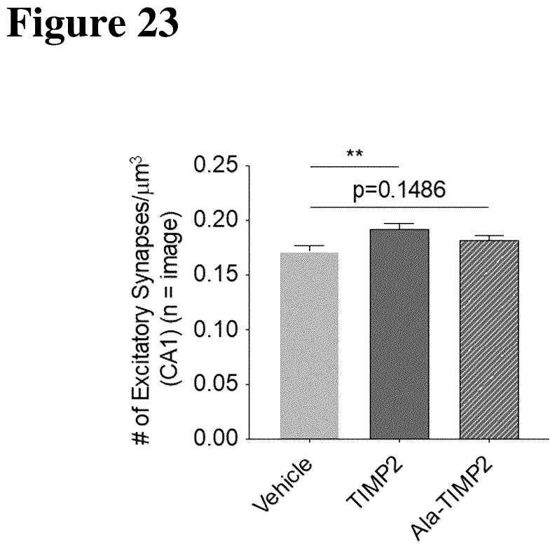

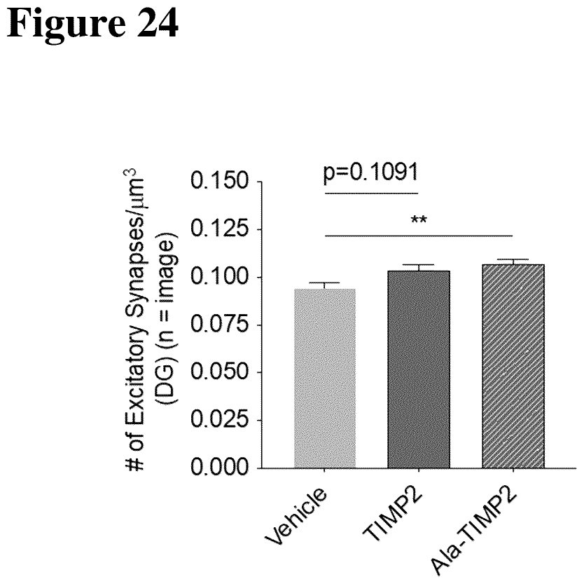

Excitatory synapses were significantly increased in the CA1 hippocampal region with TIMP2-hIgG4 treatment (FIG. 21) and increased in the dentate gyrus (DG) with both TIMP2 and TIMP2-hIgG4 treatment (FIG. 22). Excitatory synapses were significantly increased in the CA1 hippocampal region with TIMP2 treatment (FIG. 23) and increased in the dentate gyrus (DG) with Ala-TIMP2 treatment (FIG. 24).

The increase in excitatory synapses in the hippocampus could be one of the underlying mechanisms of improved cognition in the animals. Further, the DG may be more susceptible to synaptic improvements with systemic treatment. Improvement in excitatory synapses with Ala-TIMP2 treatment also suggests that MMP inhibitory activity is not necessary for improvement and that abrogation of the MMP inhibitory activity of TIMP2 through such recombinant protein constructs can help to avoid unwanted side effects commonly reported with MMP inhibitors such as musculoskeletal pain and inflammation (Vandenbroucke R E, et al. and Fingleton B, supra).

5. Brain Penetrance of TIMP2 Constructs

Male C57BL/6 mice at 22.5 months of age were used to measure the brain penetrance of TIMP2 and TIMP2-hIgG4 following a single IP dose of 1 mg/kg. Three (3) mice were dosed with TIMP2 or TIMP2-hIgG4 and sacrificed at 30 minutes, 2 hours, or 6 hours post-dose. An additional 2 mice were dosed with vehicle and sacrificed at 30 minutes post-dose for a control. Hemibrain lysates were homogenized in Tissue Extraction Reagent I (FNN0071, Thermo Scientific). Tissue was homogenized using the Bead Ruptor; homogenates were centrifuged at max speed (21,330×g) for 20 minutes at 4° C.; then supernatants were collected for subsequent analysis of the soluble fraction. Human TIMP2 levels in the brain were detected using a human TIMP-2 DuoSet ELISA (DY971, R&D Systems) and DuoSet Ancillary Reagent Kit 2 (DY008, R&D Systems). All samples were run in duplicate and the ELISA plates at a dilution of 1:2 and were read on a BMG LABTECH CLARIOstar plate reader at 450 nm and wavelength correction set at 540 nm.

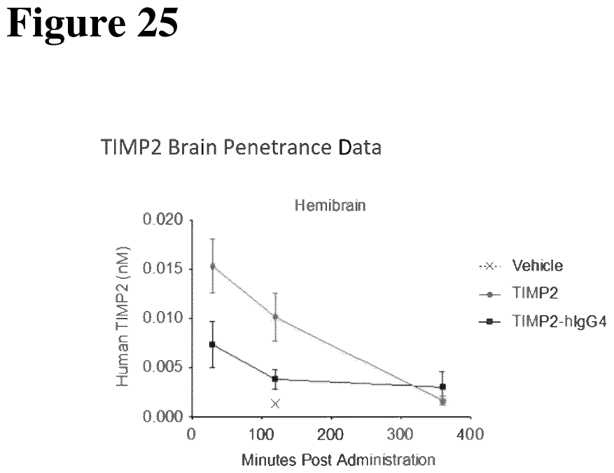

FIG. 25 shows that both TIMP2 and TIMP2-hIgG4 are detectable in brain lysate up to 2 hours following a single dose of 1 mg/kg IP. The brain penetrance of TIMP2 has previously been reported (Castellano 2017, supra) and expected. However, the brain penetrance of TIMP2-hIgG4 was surprisingly unexpected due to its large molecular weight and fusion with hIgG4 which as we have shown, dimerizes and in turn results in an even larger sized protein complex. Additionally, there are active mechanisms that prevent IgG from getting into the brain in appreciable amounts (see, e.g., Yu Y J et al., Neurotherapeutics, 10(3):459-72 (2013); Pardridge W M, Expert Opin Drug Deliv, 12(2):207-22 (2015); and Bein-Ly N et al., Neuron, 88(2):P289-97 (2015)). These data suggest there may be an active mechanism for shuttling TIMP2 into the brain and that this recombinant protein construct with a substantially longer half-life compared to TIMP2 can be peripherally administered at a lower dose frequency yet cross directly into the brain.

1. A recombinant human tissue inhibitor of metalloprotease 2 (TIMP-2) protein comprising one or more modifications relative to native human TIMP-2.

2. The recombinant human TIMP-2 protein according to claim 1, wherein the protein comprises a signal peptide not present in native human TIMP-2.

3. The recombinant human TIMP-2 protein according to claim 2, wherein the signal peptide comprises a human interleukin-2 (hIL-2) leader sequence.

4. The recombinant human TIMP-2 protein according to claim 1, wherein the protein comprises a sequence of an immunoglobulin Fc region.

5. The recombinant human TIMP-2 protein according to claim 4, wherein the sequence of an immunoglobulin Fc region comprises a hIgG4 sequence.

6. The recombinant human TIMP-2 protein according to claim 1, wherein the protein does not comprise the full-length human TIMP-2 sequence.

7. The recombinant human TIMP-2 protein according to claim 1, wherein protein comprises an amino acid insertion.

8. The recombinant human TIMP-2 protein according to claim 7, wherein the insertion is internal to the protein.

9. The recombinant human TIMP-2 protein according to claim 8, wherein the insertion is before amino acid number 27 of the native human TIMP-2 sequence.

10. The recombinant human TIMP-2 protein according to claim 9, wherein the insertion comprises alanine.

11. A nucleic acid encoding a protein according to claim 1.

12. A vector comprising a nucleic acid according to claim 11.

13. A method of treating an adult mammal for an aging-associated condition, the method comprising: enhancing a TIMP activity in the mammal in a manner sufficient to treat the adult mammal for the aging-associated condition by administering to the mammal a composition comprising a protein, nucleic acid or vector according to claim 1.

14. The method according to claim 13, wherein the mammal is a primate.

15. The method according to claim 13, wherein the adult mammal is an elderly mammal.

16. The method according to claim 15, wherein the primate is a human.

17. The method according to claim 13, wherein the aging-associated condition comprises a cognitive impairment or a cognitive decline disease condition


SEQUENCE LISTING (38 sequences)

SEQ ID NO: 1 - protein, 214 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 2 - protein, 146 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGC

SEQ ID NO: 3 - protein, 215 aa, synthetic construct
MYRMQLLSCIALSLALVTNSACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 4 - protein, 88 aa, synthetic construct
MYRMQLLSCIALSLALVTNSECKITRCPMIPCYISSPDECLWMDWVTEKNINGHQAKFFACIKRSDGSCAWYRGAAPPKQEFLDIEDP

SEQ ID NO: 5 - protein, 222 aa, synthetic construct
MYRMQLLSCIALSLALVTNSHHHHHHHHCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 6 - protein, 222 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPHHHHHHHH

SEQ ID NO: 7 - protein, 420 aa, synthetic construct
MYRMQLLSCIALSLALVTNSSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 8 - protein, 420 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 9 - protein, 439 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSL

SEQ ID NO: 10 - protein, 154 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCHHHHHHHH

SEQ ID NO: 11 - protein, 96 aa, synthetic construct
MYRMQLLSCIALSLALVTNSECKITRCPMIPCYISSPDECLWMDWVTEKNINGHQAKFFACIKRSDGSCAWYRGAAPPKQEFLDIEDPHHHHHHHH

SEQ ID NO: 12 - protein, 355 aa, synthetic construct
MYRMQLLSCIALSLALVTNSCSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCGVSSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 13 - protein, 294 aa, synthetic construct
MYRMQLLSCIALSLALVTNSECKITRCPMIPCYISSPDECLWMDWVTEKNINGHQAKFFACIKRSDGSCAWYRGAAPPKQEFLDIEDPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 14 - protein, 226 aa, synthetic construct
MYRMQLLSCIALSLALVTNSSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 15 - protein, 245 aa, synthetic construct
MYRMQLLSCIALSLALVTNSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSL

SEQ ID NO: 16 - protein, 246 aa, synthetic construct
MYRMQLLSCIALSLALVTNSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 17 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 18 - protein, 220 aa, synthetic construct
MGAAARTLRLALGLLLLATLLRPADAASCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 19 - protein, 228 aa, synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPHHHHHHHH

SEQ ID NO: 20 - protein, 426 aa, synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 21 - protein, 430 aa, synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 434 aa, synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 23 - protein, 20 aa, Homo sapiens
MYRMQLLSCIALSLALVTNS

SEQ ID NO: 24 - protein, 26 aa, Homo sapiens
MGAAARTLRLALGLLLLATLLRPADA

SEQ ID NO: 25 - protein, 194 aa, Homo sapiens
CSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 26 - protein, 126 aa, Homo sapiens
CSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGC

SEQ ID NO: 27 - protein, 68 aa, Homo sapiens
ECKITRCPMIPCYISSPDECLWMDWVTEKNINGHQAKFFACIKRSDGSCAWYRGAAPPKQEFLDIEDP

SEQ ID NO: 28 - protein, 205 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSL

SEQ ID NO: 29 - protein, 206 aa, synthetic construct
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 30 - protein, 20 aa, Homo sapiens
ESKYGPPCPPCPAPEFLGGP

SEQ ID NO: 31 - protein, 8 aa, synthetic construct
HHHHHHHH

SEQ ID NO: 32 - protein, 210 aa, Homo sapiens
PSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 214 aa, Homo sapiens
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 34 - protein, 400 aa, synthetic construct
ACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSL

SEQ ID NO: 35 - protein, 425 aa, synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSL

SEQ ID NO: 36 - protein, 221 aa, synthetic construct
MGAAARTLRLALGLLLLATLLRPADAACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 37 - protein, 427 aa, synthetic construct
MGAAARTLRLALGLLLLATLLRPADAACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 38 - protein, 234 aa, synthetic construct
MYRMQLLSCIALSLALVTNSVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK